(19) United States
(12) Patent Application Publication
(10) Pub. No.: US 2009/0061515 A1
(43) Pub. Date: Mar. 5, 2009

(54) Methods of inhibiting or suppressing cellular proliferation

(76) Inventors: Michael Eric Benz, Ramsey, MN (US); Timothy H. Robinson, Savage, MN (US); Lian Leon Luo, Shoreview, MN (US); Jesus Casas-Bejar, Brooklyn Park, MN (US); Maura Donovan, St. Paul, MN (US); Krystal King, Richfield, MN (US)

Correspondence Address: MUETING, RAASCH & GEBHARDT, P.A., P.O. BOX 581336, MINNEAPOLIS, MN, 55458-1336, US

(21) Appl. No.: 11/897,085

(22) Filed: Aug. 29, 2007

(51) Int. Cl. C12N 5/06

(52) U.S. Cl. 435/375

(57) ABSTRACT

Methods of inhibiting or suppressing cellular proliferation are disclosed that include delivering at least one antiproliferative agent into or proximate a cell. In certain embodiments, the antiproliferative agent(s) are hydrolysis products of a biodegradable polymer (e.g., a polyketal polymer).

### BACKGROUND

Antiproliferative agents are compounds that can inhibit or suppress cellular proliferation (i.e., growth and multiplication of the cells). The use of antiproliferative agents to treat or prevent a wide variety of proliferative (e.g., hyperproliferative) conditions and/or diseases has been disclosed. Examples of such conditions and/or diseases include, but are not limited to, hyperplasia of soft and hard tissues, malignant tumors such as cancer, skin keloids, fibrosis, and surgical adhesions. Antiproliferative agents can also be used to prevent cell proliferative processes resulting in in-stent restenosis (e.g., associated with coronary stents), pannus overgrowth on prosthetic heart valves (e.g., associated with sewing rings), urethral stenosis (e.g., associated with prostatic hyperplasia), and stenosis associated with surgical anastomosis.

While many agents (e.g., drugs) are known having strong antiproliferative potential, their mechanism of action typically involves cytotoxicity. The extensive side effects observed during and after anticancer chemotherapy are illustrative of the use of antiproliferative agents having significant potential for cytotoxicity. Cytostatic agents are compounds that can inhibit or suppress cellular proliferation (i.e., growth and multiplication of the cells) without compromising the cell's viability and functionality. Few drugs have proven to have an effective cytostatic window with minimal cytotoxic potential.

New methods and/or agents for inhibiting or suppressing cellular proliferation are needed.

### SUMMARY

Methods of inhibiting or suppressing cellular proliferation are disclosed herein. In one embodiment, the method includes delivering into or proximate a cell at least one antiproliferative agent selected from the group consisting of: a compound of the formula (Formula I):

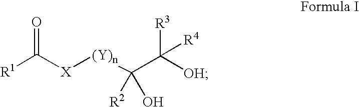

a compound of the formula (Formula II):

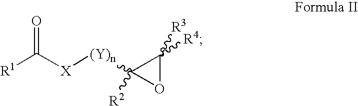

a compound of the formula (Formula III):

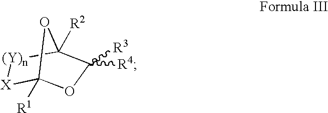

and combinations thereof; wherein: each X independently represents NR⁵, CR⁵R⁶, SiR⁵R⁶, S, a sulfur-bonded group, a phosphorus-bonded group, or

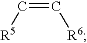

each Y independently represents O, NR⁵, CR⁵R⁶, SiR⁵R⁶, S, a sulfur-bonded group, a phosphorus-bonded group, or

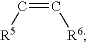

each n is independently from 0 to 5; each R¹ independently represents an organic group; each R², R³, R⁴, R⁵, and R⁶ independently represents H or an organic group; and R¹, R², R³, R⁴, R⁵, and/or R⁶ can optionally be joined to each other to form one or more rings.

In another embodiment, the method includes locating at least one polymer proximate a tissue; allowing the at least one polymer to hydrolyze to form at least one antiproliferative agent of Formula I, Formula II, and/or Formula III; and delivering the at least one antiproliferative agent into or proximate a cell.

In another embodiment, the method includes: providing a medical device including at least one biodegradable polymer; positioning the at least one biodegradable polymer proximate a tissue; allowing the at least one biodegradable polymer to biodegrade to form at least one antiproliferative agent of Formula I, Formula II, and/or Formula III; and delivering the at least one antiproliferative agent into or proximate a cell.

In another embodiment, the method including delivering into or proximate a cell at least one antiproliferative agent selected from the group consisting of: a ketal and/or a hemiketal of a compound of Formula I and/or Formula II.

In yet another embodiment, the method includes delivering into or proximate a cell at least one prodrug that can release an antiproliferative agent of Formula I, Formula II, and/or Formula III.

The terms “comprises” and variations thereof do not have a limiting meaning where these terms appear in the description and claims.

As used herein, “a,” “an,” “the,” “at least one,” and “one or more” are used interchangeably.

As used herein, the term “or” is generally employed in the sense as including “and/or” unless the context of the usage clearly indicates otherwise.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a graphical representation of the cytotoxicity of normal human dermal fibroblast (NHDF) cells exposed to various concentrations of 5,6-dihydroxy-hexan-2-one for 24 hours and 48 hours as described in Example 3.

FIGS. 2-6 are pictures that visually display the physical cellular response of normal human dermal fibroblast (NHDF) cells to various concentrations of 5,6-dihydroxy-hexan-2-one as described in Example 3.

FIGS. 7-16 are pictures that visually display the physical cellular response of normal human dermal fibroblast (NHDF) cells to various concentrations of 5,6-dihydroxy-hexan-2-one as described in Example 4.

FIG. 17 is a graphical representation of normal human dermal fibroblast (NHDF) cell numbers measured after 48 hours exposure to different concentrations of 5,6-dihydroxy-hexan-2-one as described in Example 5.

FIG. 18 is a graphical representation of coronary artery smooth muscle cell (CASMC) numbers measured after 48 hours exposure to different concentrations of 5,6-dihydroxy-hexan-2-one as described in Example 5.

FIG. 19 is a graphical representation of viable coronary artery smooth muscle cell (CASMC) numbers measured after 48 hours exposure to different concentrations of 5,6-dihydroxy-hexan-2-one as described in Example 5.

FIGS. 20(a)-(l) are pictures that visually display the migration of coronary artery smooth muscle cells (CASMC) (FIGS. 20(a)-(f)) and normal human dermal fibroblast (NHDF) cells (FIGS. 20(g)-(l)) exposed to various concentrations of 5,6-dihydroxy-hexan-2-one after 48 hours exposure, then cultured an additional 72 hours after removal of the 5,6-dihydroxy-hexan-2-one as described in Example 5.

FIG. 21 is a graphical representation of human Glioblastoma/Astrocytoma U87 cell numbers measured after being exposed to 5,6-dihydroxy-hexan-2-one for 48 hours as described in Example 6. Time 0 refers to the replacement of the test agent with supplemented growth media.

FIG. 22 is a graphical representation of human umbilical vein endothelial cell (HUVEC) numbers measured after being exposed to 5,6-dihydroxy-hexan-2-one for 48 hours as described in Example 6. Time 0 refers to replacement of the test agent with supplemented growth media.

FIGS. 23(a)-(l) are pictures that visually display the migration over time of human umbilical vein endothelial cells (HUVEC) after 48 hours exposure to various concentrations of 5,6-dihydroxy-hexan-2-one as described in Example 6.

FIGS. 24(a)-(l) are pictures that visually display the migration over time of human Glioblastoma/Astrocytoma U87 cells after 48 hours exposure to various concentrations of 5,6-dihydroxy-hexan-2-one as described in Example 6.

FIGS. 25(a) and (b) are graphical illustrations of cell counts and cell viability, respectively, for human coronary artery endothelial cells (HCAEC) exposed to 5,6-dihydroxy-6-phenyl-hexan-2-one for 24 hours and 48 hours, then allowed to grow for 96 hours after removal of 5,6-dihydroxy-6-phenyl-hexan-2-one as described in Example 7.

FIGS. 26(a) and (b) are graphical illustrations of cell counts and cell viability, respectively, for human coronary artery endothelial cells (HCAEC) exposed to 5,6-dihydroxy-heptan-2-one for 24 hours and 48 hours, then allowed to grow for 96 hours after removal of 5,6-dihydroxy-heptan-2-one as described in Example 7.

FIGS. 27(a) and (b) are graphical illustrations of cell counts and cell viability, respectively, for human coronary artery endothelial cells (HCAEC) exposed to 5,6-dihydroxy-5-methyl-hexan-2-one for 24 hours and 48 hours, then allowed to grow for 96 hours after removal of 5,6-dihydroxy-5-methyl-hexan-2-one as described in Example 7.

FIGS. 28(a) and (b) are graphical illustrations of cell counts and cell viability, respectively, for human coronary artery endothelial cells (HCAEC) exposed to 2-(2′,3′-dihydroxypropyl)-cyclohexanone for 24 hours and 48 hours, then allowed to grow for 96 hours after removal of 2-(2′,3′-dihydroxypropyl)-cyclohexanone as described in Example 7.

FIGS. 29(a) and (b) are graphical illustrations of cell counts and cell viability, respectively, for human coronary artery endothelial cells (HCAEC) exposed to 2,3-dihydroxypropyl acetate for 24 hours and 48 hours, then allowed to grow for 96 hours after removal of 2,3-dihydroxypropyl acetate as described in Example 7.

FIGS. 30(a) and (b) are graphical illustrations of cell counts and cell viability, respectively, for human coronary artery endothelial cells (HCAEC) exposed to 5,6-dihydroxy-hexan-2-one for 24 hours and 48 hours, then allowed to grow for 96 hours after removal of 5,6-dihydroxy-hexan-2-one as described in Example 7.

### DETAILED DESCRIPTION OF ILLUSTRATIVE EMBODIMENTS

The present disclosure provides methods of inhibiting or suppressing cellular proliferation. The methods include the delivery of at least one antiproliferative agent as described herein into or proximate a cell (e.g., into or near a tissue, region, or organ). In certain embodiments, the antiproliferative agent (e.g., a cytostatic agent) can inhibit or suppress cellular proliferation without exhibiting cytotoxic effects (e.g., cell death). In certain embodiments, the inhibition or suppression of cellular proliferation upon delivery of the at least one antiproliferative agent into or proximate the cell is irreversible. Preferably the antiproliferative agent can inhibit or suppress cellular proliferation upon delivery into or proximate a wide variety of cell types.

The antiproliferative agent can be a compound of the formula (Formula I):

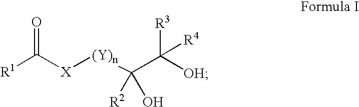

a compound of the formula (Formula II):

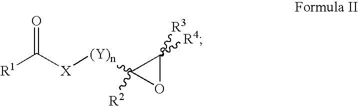

a compound of the formula (Formula III):

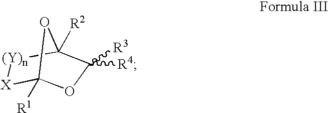

or a combination thereof; wherein: each X independently represents NR⁵, CR⁵R⁶, SiR⁵R⁶, S, a sulfur-bonded group (i.e., an organic or inorganic group bonded through sulfur such as, for example, S(O), S(O)₂, or the like), a phosphorus-bonded group (i.e., an organic or inorganic group bonded through phosphorus such as, for example, PR or PR₃, where R is an organic group, or the like), or

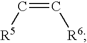

each Y independently represents O, NR⁵, CR⁵R⁶, SiR⁵R⁶, S, a sulfur-bonded group, a phosphorus-bonded group, or

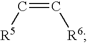

each n is independently from 0 to 5; each R¹ independently represents an organic group; each R², R³, R⁴, R⁵, and R⁶ independently represents H or an organic group; and R¹, R², R³, R⁴, R⁵, and/or R⁶ can optionally be joined to each other to form one or more rings. The wavy bonds in the formulas herein are used to indicate unspecified stereochemistry. It should be noted that antiproliferative agents of Formula I, Formula II, and Formula III are intended to include any dimers and/or trimers of the indicated compounds that may exist either independently or in equilibrium with compounds of the indicated formulas.

In certain embodiments, each X and Y independently represents CR⁵R⁶; each n is 1; each R¹ independently represents a C₁-C₁₀ organic group; each R², R³, R⁴, R⁵, and R⁶ independently represents H or a C1-C10 organic group; and R¹ and R⁵ can optionally be joined to each other to form a ring.

In other certain embodiments, each X and Y independently represents CR⁵R⁶; each n is 1; each R¹ independently represents a phenyl group (and preferably a phenyl ring) or a C1-C4 aliphatic or alicyclic group (and preferably a C1-C4 aliphatic or alicyclic moiety); each R², R³, R⁴, R⁵, and R⁶ independently represents H, a phenyl group, or a C1-C4 aliphatic or alicyclic group (and preferably H, a phenyl group, or a C1-C4 aliphatic or alicyclic moiety); and R¹ and R⁵ can optionally be joined to each other to form a five- or six-membered ring.

In one preferred embodiment, the antiproliferative agent is 5,6-dihydroxy-hexan-2-one (i.e., Formula I, wherein R¹=methyl; X═Y═CH₂; n=1; and R²═R³═R⁴═H). In another preferred embodiment, the antiproliferative agent is 5,6-dihydroxy-heptan-2-one (i.e., Formula I, wherein R¹=methyl; X═Y═CH₂; n=1; R²═R³═H; and R⁴=methyl). In another preferred embodiment, the antiproliferative agent is 5,6-dihydroxy-5-methyl-hexan-2-one (i.e., Formula I, wherein R¹=methyl; X═Y═CH₂; n=1; and R²=methyl; and R³═R⁴═H). In another preferred embodiment, the antiproliferative agent is 5,6-dihydroxy-6-phenyl-hexan-2-one (i.e., Formula I, wherein R¹=methyl; X═Y═CH₂; n=1; R²═R³═H; and R⁴=phenyl). In another preferred embodiment, the antiproliferative agent is 4,5-dihydroxy-1-phenyl-pentan-1-one (i.e., Formula I, wherein R¹=phenyl; X═Y═CH₂; n=1; and R²═R³═R⁴═H). In another preferred embodiment, the antiproliferative agent is 2-(2′,3′-dihydroxypropyl)-cyclohexanone (i.e., Formula I, wherein R¹ and X are joined together to form a cyclohexanone ring; Y═CH₂; n=1; and R²═R³═R⁴═H). In another preferred embodiment, the antiproliferative agent is 2,3-dihydroxypropyl acetate (i.e., Formula I, wherein R¹=methyl; X=oxygen; Y═CH₂; n=1; and R²═R³═R⁴═H).

In another preferred embodiment, the antiproliferative agent is 5,6-epoxy-hexan-2-one (i.e., Formula II, wherein R¹=methyl; X═Y═CH₂; n=1; and R²═R³═R⁴═H).

In some embodiments, the antiproliferative agent can be a ketal or hemiketal of a compound of Formula I or Formula II, and/or a hydrolysis product thereof. For example, the ketone group in Formula I or Formula 11 can react with one or more alcohols (including diols or polyols) to form a ketal or hemiketal. For another example, the diol group of Formula I can react with a ketone or aldehyde to form a cyclic ketal or acetal. For even another example, a cyclic hemiketal can be formed by the cyclization of a compound of Formula I. A cyclic hemiketal formed by the cyclization of a compound of Formula I can be represented, for example, by the formulas

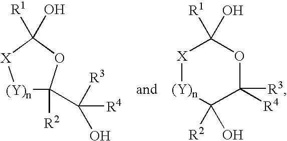

wherein each X, Y, n, R¹, R², R³, and R⁴ is defined as disclosed herein above for compounds of Formula I.

In certain embodiments, the antiproliferative agent can be a prodrug that can release (e.g., upon metabolism and/or hydrolysis) a compound of Formula I, Formula II, and or Formula III. For example, a ketal of a compound of Formula I (as described herein) can be hydrolyzed to release a compound of Formula I.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for monomers and polymers of this invention are those that do not interfere with the ring opening polymerization reaction disclosed herein. In the context of the present invention, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The antiproliferative agent can be delivered into or proximate the cell by a wide variety of methods. For example, in some embodiments the antiproliferative agent can be delivered topically, by inhalation, by contact with mucuous tissues, by injection, and combinations thereof. For example, in some embodiments the antiproliferative agent can be delivered directly to the cell (e.g., as a suspension, dispersion, or emulsion) by injection via a needle or catheter. Other embodiments include delivery of the antiproliferative agent in semisolid and/or solid formulations designed to provide continuous and/or controlled release of the antiproliferative agent into the tissue-biomaterial interface or surrounding tissue. Other applications include the use of antiproliferative agents as components of cell, drug, and/or gene therapy formulations, as adjuvants for therapeutic potential. Further applications include, for example, limiting tumor growth.

In certain embodiments, the antiproliferative agent can be delivered in suspension or solution into the extracellular space from which it can be diffused, distributed, contacted with, and/or internalized by cells and/or tissue. In another embodiment, the antiproliferetive agent can be delivered as a component of a hydrogel that solidifies upon contact with living tissue for release to targeted cells and/or tissues. In yet another embodiment, the antiproliferative agent can be delivered in a solid form (e.g., films, pellets, microspheres, and the like, that include, among other things, the antiproliferative agent), for release to targeted cells and/or tissues as biodegradation occurs. In yet another embodiment, the antiproliferative agent can be delivered using a liposome (e.g., by diffusion from the liposome). In addition to the above-described embodiments, the antiproliferative agent can be combined with at least one cell type, either to optimize the delivery of cells or to protect the implanted cells from surrounding physiological and/or pathological events such as inflammation and/or rejection, preferably increasing the therapeutic potential of cell-based therapies. In addition to the above-described embodiments, the antiproliferative agent can be combined with at least one cell type, at least one therapeutic drug, and/or at least one molecular material designed to modify the expression of a certain gene affecting the etiology of a given therapy.

In some embodiments, at least one antiproliferative agent as described herein can be disposed in a polymer, and the polymer can be located proximate a cell and allowed to deliver the at least one antiproliferative agent into or proximate the cell. As used herein, the term “disposed” is intended to be broadly interpreted as inclusive of dispersed, dissolved, suspended, or otherwise contained at least partially therein or thereon. In such embodiments, the polymer can deliver the at least one antiproliferative agent by a variety of mechanisms including, for example, delivery of the at least one antiproliferative agent from pores in the polymer, diffusion of the at least one antiproliferative agent through the polymer, delivery of the at least one antiproliferative agent through degradation of the polymer, or combinations thereof.

In some other embodiments, a medical device including at least one antiproliferative agent as described herein, and the medical device can be located proximate a cell and allowed to deliver the at least one antiproliferative agent into or proximate the cell. Optionally, the medical device including the at least one antiproliferative agent can include a polymer having the at least one antiproliferative agent disposed therein.

A wide variety of polymers can be used in the methods disclosed herein including, but not limited to, polyurethanes (e.g., polyether urethanes, polyester urethanes including polycaprolactone urethanes), polyureas, polyurethane-ureas, polyesters (e.g., polyethylene terephthalate), poly(beta-aminoesters), polycarbonates, poly(meth)acrylates, polysulfones, polyimides, polyamides, epoxies, polyacetals, polyketals, polyorthoesters, vinyl polymers, polyanhydrides, polytriazoles, silicone rubber, natural rubber, rubber latex, synthetic rubbers, polyether-polyamide block copolymers, polyester-polyether copolymers, and combinations and/or copolymers thereof. Exemplary polyesters include, for example, linear aliphatic polyester homopolymers (e.g., polyglycolide, polylactide, polycaprolactone, and polyhydroxybutyrate) and copolymers (e.g., poly(glycolide-co-lactide), poly(glycolide-co-caprolactone), poly(glycolide-co-trimethylenecarbonate), poly(lactic acid-co-lysine), poly(lactide-co-urethane), poly(ester-co-amide)). Polymers used in the methods disclosed herein can be biostable or biodegradable.

Polymers used in the methods disclosed herein having antiproliferative agents disposed therein can be prepared by a wide variety of methods known in the art. For example, such compositions can be prepared by solution processing, milling, extruding, polymerizing components in the presence of an antiproliferative agent, and combinations thereof.

In still other embodiments, at least one polymer can be located proximate a tissue and allowed to hydrolyze to form at least one antiproliferative agent, which can be delivered into or proximate a cell. The at least one polymer can be a polyester, a polyorthoester, a polyketal (e.g., as described in U.S. application Ser. No. 11/706,508, filed 15 Feb. 2007), or a combination thereof that can form at least one antiproliferative agent upon hydrolysis (e.g., biodegradation).

Polymers used in the methods disclosed herein can be used as, for example, tissue bulking agents, tissue replacement agents, tissue repair agents, surgical void fillers, agents used to prevent surgical adhesions, or combinations thereof.

Certain polyketals are also known to be biodegradable polymers. As used herein, a “polyketal” refers to a homo- or co-polymer that includes two or more (i.e., a plurality) of ketal repeat units. As used herein, a “ketal” repeat unit is a unit including a ketal-containing group that is repeated in the polymer at least once. A ketal group is a group that includes an —C—O—C(M)(N)—O—C— functionality with the proviso that neither M nor N is hydrogen (e.g., an acetal-containing group) or oxygen (e.g., an orthoester-containing group). Typically, M and N are attached to the carbon atom of the ketal group via a carbon-carbon bond.

Exemplary polyketal polymers include two or more cyclic oxygen-containing repeat units selected from the group consisting of: a repeat unit of the formula (Formula IV):

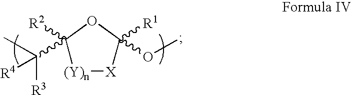

a repeat unit of the formula (Formula V):

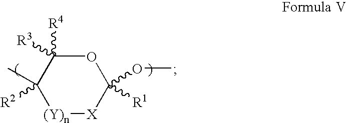

and combinations thereof; wherein: each X independently represents NR⁵, CR⁵R⁶, SiR⁵R⁶, S, a sulfur-bonded group, a phosphorus-bonded group, or

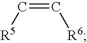

each Y independently represents O, NR⁵, CR⁵R⁶, SiR⁵R⁶, S, a sulfur-bonded group, a phosphorus-bonded group, or

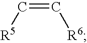

each n is independently from 0 to 5; each R¹ independently represents an organic group; each R², R³, R⁴, R⁵, and R⁶ independently represents H or an organic group; and R¹, R², R³, R⁴, R⁵, and/or R⁶ can optionally be joined to each other to form one or more rings.

In certain embodiments, each X and Y independently represents CR⁵R⁶; each n is 1; each R¹ independently represents a C1-C10 organic group; each R², R³, R⁴, R⁵, and R⁶ independently represents H or a C1-C10 organic group; and R¹ and R⁵ can optionally be joined to each other to form a ring.

In other certain embodiments, each X and Y independently represents CR⁵R⁶; each n is 1; each R¹ independently represents a phenyl group (and preferably a phenyl ring) or a C1-C4 aliphatic or alicyclic group (and preferably a C1-C4 aliphatic or alicyclic moiety); each R², R³, R⁴, R⁵, and R⁶ independently represents H, a phenyl group, or a C1-C4 aliphatic or alicyclic group (and preferably H, a phenyl group, or a C1-C4 aliphatic or alicyclic moiety); and R¹ and R⁵ can optionally be joined to each other to form a five- or six-membered ring.

In some embodiments, the polyketal polymer can further include repeat units selected from the group consisting of crosslinkable repeat units, crosslinked repeat units, repeat units having imagable groups, repeat units having latent reactive sites, and combinations thereof. In certain embodiments, the polyketal polymer can further include repeat units selected from the group consisting of alpha-hydroxy alkanoates, beta-hydroxy alkanoates, gamma-hydroxy alkanoates, delta-hydroxy alkanoates, epsilon-hydroxy alkanoates, glycols, carbonates, acetals, and combinations thereof.

The polyketal polymers disclosed herein can include a single cyclic oxygen-containing repeat unit (i.e., a homopolymer), or two or more different repeat units (i.e., a copolymer). In such copolymers, the two or more different repeat units can all be different cyclic oxygen-containing repeat units of Formula IV and/or Formula V, or alternatively, one or more cyclic oxygen-containing repeat units of Formula IV and/or Formula V in combination with one or more repeat units that are not of Formula IV or Formula V (e.g., lactide repeat units, glycolide repeat units, butyrolactone repeat units, valerolactone repeat units, caprolactone repeat units, cyclic carbonate repeat units such as trimethylene carbonate and 1,2-O-isopropylidene-[D]-xylofuranose-3,5-cyclic carbonate, cyclic ether repeat units such as ethylene oxide, cyclic acetals such as 1,3-dioxolane, and combinations thereof). The polymers disclosed herein can be linear polymers, crosslinkable polymers, and/or crosslinked polymers.

Optionally, the polyketal polymer can be a copolymer. The copolymer can be a random copolymer, an alternating copolymer, a block copolymer, a graft copolymer, or a combination thereof. For embodiments in which the copolymer is a block copolymer, at least one block of the block copolymer can be a polyketal block including the two or more repeat units selected from the group consisting of repeat units of Formula IV, repeat units of Formula V, and combinations thereof. In certain of these embodiments, at least one other block of the block copolymer includes repeat units such as alpha-hydroxy alkanoates, beta-hydroxy alkanoates, gamma-hydroxy alkanoates, delta-hydroxy alkanoates, epsilon-hydroxy alkanoates, carbonates, acetals, or combinations thereof. In certain of these embodiments, at least one other block of the block copolymer is a polyorthoester block. In certain of these embodiments, at least one other block of the block copolymer is a poly(alkyleneglycol) block including alkylene glycol repeat units.

Notably the presently disclosed polyketals include polymers that are not converted under physiological conditions to acidic products. Further, the present invention provides polyketal polymers that can biodegrade at a sufficiently high rate to enable them to be considered for use in specific applications. As used herein, “biodegradable” and “bioerodible” are used interchangeably and are intended to broadly encompass materials including, for example, those that tend to break down upon exposure to physiological environments. Notably, R¹ in each of the polyketal repeating units disclosed herein represents an organic group which is advantageous in providing polymers with useful biodegradability. In contrast, polysaccharides are structures in which R¹ represents hydrogen. Although polysaccharides are useful biomaterials (e.g., useful in biomedical applications), they typically do not rapidly biodegrade in physiologic environments.

Typically and preferably, the polyketal polymers disclosed herein are biodegradable. Typically, the average molecular weight (and preferably the weight average molecular weight) of the polymers disclosed herein is at least 1000 Daltons, and sometimes at least 2000 Daltons, 5,000 Daltons, or even 10,000 Daltons or more. Average molecular weights of the polymers disclosed herein can be as high as desired for specific applications. Typically, the average molecular weight (and preferably the weight average molecular weight) of the polymers disclosed herein is at most 10,000,000 Daltons, and sometimes at most 5,000,000 Daltons, 2,000,000 Daltons, or even 1,000,000 Daltons. Typically the polydispersity index (PDI) of the polymers disclosed herein is at most 3, and sometimes at most 2.5, and other times at most 2.0.

For certain applications, polymers used in the methods disclosed herein (e.g., polyketal polymers) can be blended with another polymer (e.g., the same or different) to provide the desired physical and/or chemical properties. For example, two polyketal polymers having different molecular weights can be blended to optimize the release rate of a biologically active agent. For another example, two polyketal polymers having different repeat units can be blended to provide desired physical and/or chemical properties. For even another example, a polyketal polymer can be blended with another polymer that is not a polyketal polymer to provide desired physical and/or chemical properties.

Polymers used in the methods disclosed herein can be used in various combinations for various applications. They can be used for replacements for nucleus pulposis in intervertebral disc repair procedures. They can be used as tissue adhesives or sealants. They can be used as surgical void fillers, for example, in reconstructive or cosmetic surgery (e.g., for filling a void after tumor removal). They can be used to repair aneurysms, hemorrhagic stroke or other conditions precipitated by failure of a blood vessel. They can be used to prevent surgical adhesions. They can be used to limit tumor growth.

Polymers used in the methods disclosed herein can be used in injectable compositions. Such injectable compositions could be used, for example, as void fillers (e.g., in cosmetic or reconstructive surgery, such as serving as a replacement for the nucleus pulposis) or as an injectable drug delivery matrix.

In some embodiments, no additives would be needed to form an injectable composition. In some embodiments, one or more polymers can be combined with a solvent such as N-methyl-2-pyrrolidone or dimethylsulfoxide (DMSO), which are fairly biocompatible solvents. The solvent can diffuse away after injection and the polymer can remain in place. Such injectable materials can be applied to a desired site (e.g., a surgical site) using a syringe, catheter, applicator, or by hand.

Also, injectable compositions could include crosslinkers (such as diacrylates), plasticizers (such as triethyl citrate), lipids (soybean oil), poly(ethylene glycol) (including those with the ends blocked with methyls or similar groups), silicone oil, partially or fully fluorinated hydrocarbons, N-methyl-2-pyrrolidone, or mixtures thereof.

Polymers used in the methods disclosed herein can be used in combination with a variety of particulate materials. For example, they can be used with moisture curing ceramic materials (e.g., tricalcium phosphate) for vertebroplasty cements, bone void filling (due to disease such as cancer or due to fracture). They can be used in combination with inorganic materials such as hydroxyapatite to form pastes for use in bone healing, sealing, filling, repair, and replacement. They can be used as or in combination with polymer microspheres that can be reservoirs for a biologically active agent such as a protein, DNA plasmid, RNA plasmid, antisense agent, etc.

Alternatively, polymers used in the methods disclosed herein can be used in combination with other materials to form a composite (e.g., a polymer having an additive therein). In addition to the polymers used in the methods disclosed herein, composites can include a wide variety of additives, and particularly particulate additives, such as, for example, fillers (e.g., including particulate, fiber, and/or platelet material), other polymers (e.g., polymer particulate materials such as polytetrafluoroethylene can result in higher modulus composites), imaging particulate materials (e.g., barium sulfate for visualizing material placement using, for example, fluoroscopy), biologically derived materials (e.g., bone particles, cartilage, demineralized bone matrix, platelet gel, and combinations thereof), and combinations thereof. Additives can be dissolved, suspended, and/or dispersed within the composite. For particulate additives, the additive is typically dispersed within the composite.

Polymers used in the methods disclosed herein can be combined with fibers, woven or nonwoven fabric for reconstructive surgery, such as the in situ formation of a bone plate or a bone prosthesis.

In certain embodiments, one or more polymers used in the methods disclosed herein can be shaped to form a medical device, preferably a biodegradable medical device. The one or more polymers can be shaped by methods known in the art including compression molding, injection molding, casting, extruding, milling, blow molding, or combinations thereof. As used herein, a “medical device” includes devices that have surfaces that contact tissue, bone, blood, or other bodily fluids in the course of their operation, which fluids are subsequently used in patients. This can include, for example, extracorporeal devices for use in surgery such as blood oxygenators, blood pumps, blood sensors, tubing used to carry blood, and the like which contact blood which is then returned to the patient. This can also include endoprostheses implanted in blood contact in a human or animal body such as vascular grafts, stents, pacemaker leads, heart valves, and the like, that are implanted in blood vessels or in the heart. This can also include devices for temporary intravascular use such as catheters, guide wires, and the like which are placed into the blood vessels or the heart for purposes of monitoring or repair. In certain embodiments, medical devices can include biodegradable nasal and sinus stents. In certain embodiments, medical devices can include chronically removable pacemaker leads. A medical device can also be fabricated by polymerizing components in a suitable mold.

Polymers used in the methods disclosed herein can also be coated onto a substrate if desired. A coating mixture of the polymer can be prepared using solvents such as toluene, chloroform, tetrahydrofuran, perfluorinated solvents, and combinations thereof. Preferred solvents include those that can be rendered moisture-free and/or those that have no active hydrogens. The coating mixture can be applied to an appropriate substrate such as uncoated or polymer coated medical wires, catheters, stents, prostheses, penile inserts, and the like, by conventional coating application methods. Such methods include, but are not limited to, dipping, spraying, wiping, painting, solvent swelling, and the like. After applying the coating solution to a substrate, the solvent is preferably allowed to evaporate from the coated substrate.

The materials of a suitable substrate include, but are not limited to, polymers, metal, glass, ceramics, composites, and multilayer laminates of these materials. The coating may be applied to metal substrates such as the stainless steel used for guide wires, stents, catheters and other devices. Organic substrates that may be coated with the polymers used in the methods disclosed herein include, but are not limited to, polyether-polyamide block copolymers, polyethylene terephthalate, polyetherurethane, polyesterurethane, other polyurethanes, silicone, natural rubber, rubber latex, synthetic rubbers, polyester-polyether copolymers, polycarbonates, and other organic materials.

Additives that can be combined with a polymer used in the methods disclosed herein to form a composition include, but are not limited to, wetting agents for improving wettability to hydrophobic surfaces, viscosity and flow control agents to adjust the viscosity and thixotropy of the mixture to a desired level, antioxidants to improve oxidative stability of the coatings, dyes or pigments to impart color or radiopacity, and air release agents or defoamers, cure catalysts, cure accelerants, plasticizers, solvents, stabilizers (cure inhibitors, pot-life extenders), and adhesion promoters.

Optionally, the polymers used in the methods disclosed herein can include one or more biologically active agents different than the one or more antiproliferative agents described herein. As used herein, a “biologically active agent” is intended to be broadly interpreted as any agent capable of eliciting a response in a biological system such as, for example, living cell(s), tissue(s), organ(s), and being(s). Biologically active agents can include natural and/or synthetic agents. Thus, a biologically active agent is intended to be inclusive of any substance intended for use in the diagnosis, cure, mitigation, treatment, or prevention of disease or in the enhancement of desirable physical or mental development and conditions in a subject. The term “subject” as used herein is taken to include, but is not limited to, humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

A preferred class of biologically active agents includes drugs. As used herein, the term “drug” means any therapeutic agent. Suitable drugs include inorganic and organic drugs, without limitation, and include drugs that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems (including urological systems), histamine systems, and the like. Such conditions, as well as others, can be advantageously treated using compositions as disclosed herein.

Suitable drugs include, for example, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol and clocortolone.

Preferred classes of drugs include, for example, Plasmid DNA, genes, antisense oligonucleotides and other antisense agents, peptides, proteins, protein analogs, siRNA, shRNA, miRNA, ribozymes, DNAzymes and other DNA based agents, viral and non-viral vectors, liposomes, cells, stem cells, antineoplastic agents, antiproliferative agents, antithrombogenic agents, anticoagulant agents, antiplatelet agents, antibiotics, anti-inflammatory agents, antimitotic agents, immunosuppressants, growth factors, cytokines, hormones, and combinations thereof. Examples of preferred drugs are bone morphogenetic proteins (BMP) including, for example, recombinant human bone morphogenetic protein (rhBMP-2).

Suitable drugs can have a variety of uses including, but are not limited to, anticonvulsants, analgesics, antiparkinsons, antiinflammatories (e.g., ibuprofen, fenbufen, cortisone, and the like), calcium antagonists, anesthetics (e.g., benoxinate, benzocaine, procaine, and the like), antibiotics (e.g., ciprofloxacin, norfloxacin, clofoctol, and the like), antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids sympathomimetics, parasympathomimetics, tranquilizers, urinary tract drugs, vaccines, vaginal drugs, vitamins, collagen, hyaluronic acid, nonsteroidal anti-inflammatory drugs, angiotensin converting enzymes, polynucleotides, polypeptides, polysaccharides, and the like.

Certain preferred embodiments include a drug selected from the group consisting of podophyllotoxin, mycophenolic acid, teniposide, etoposide, trans-retinoic acids, 9-cis retinoic acid, 13-cis retinoic acid, rapamycin, a rapalog (e.g., Everolimus, ABT-578), camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof.

Certain preferred embodiments include a drug selected from the group consisting of salicylic acid, fenbufen, cortisone, ibuprofen, diflunisal, sulindac, difluprednate, prednisone, medrysone, acematacin, indomethacin, meloxicam, camptothecin, benoxinate, benzocaine, procaine, ciprofloxacin, norfloxacin, clofoctol, dexamethasone, fluocinolone, ketorolac, pentoxifylline, rapamycin, ABT-578, gabapentin, baclofen, sulfasalazine, bupivacaine, sulindac, clonidine, etanercept, pegsunercept, and combinations thereof.

Compositions including a biologically active agent and a polymer used in the methods disclosed herein and can be prepared by suitable methods known in the art. For example, such compositions can be prepared by solution processing, milling, extruding, polymerizing components in the presence of a biologically active agent, and combinations thereof.

Compositions including polymers used in the methods disclosed herein (e.g., with or without a biologically active agent) can further include additional components. Examples of such additional components include fillers, dyes, pigments, inhibitors, accelerators, viscosity modifiers, wetting agents, buffering agents, stabilizers, biologically active agents, polymeric materials, excipients, and combinations thereof.

Medical devices that include one or more polymers used in the methods disclosed herein and a biologically active agent can have a wide variety of uses. In such devices, the biologically active agent is preferably disposed in the one or more polymers.

For example, such devices can be used to deliver a biologically active agent to a tissue by positioning at least a portion of the device including the one or more polymers proximate the tissue and allowing the one or more polymers to biodegrade and deliver the biologically active agent disposed therein. For another example, such devices can be used to control the release rate of a biologically active agent from a medical device by disposing the biologically active agent in at least one of the one or more polymers.

The effects of the antiproliferative agents disclosed herein can be evaluated in vitro, for example, by using cultures or co-cultures of primary or commercially available cell lines. For example, endothelial cells can be isolated from aortic or coronary arteries, stem cells can be isolated from bone marrow, and myoblasts from skeletal muscle. Ultimately, adult stem cells, embryonic stem cells, somatic cells, cancer cell lines, or cells from any proliferative biopsy and/or tissue, can be utilized to evaluate the effectiveness of the antiproliferative agents disclosed herein, and to direct the application of the methods disclosed herein.

An antiproliferative agent with potential for biomedical applications can be evaluated by its effects in cell culture in vitro. When cells are exposed to a test agent (e.g., by adding into culture medium), the antiproliferative effect of the test agent can be confirmed by a reduced number of cells following a given time of exposure, in comparison to controls that do not have the test agent added into their media. In certain preferred embodiments, the test agent (e.g., a cytostatic agent) does not evidence significant signs of cytotoxicity under conditions in which the test agent shows antiproliferative effects. Furthermore, when the test agent is withdrawn from the cell culture wells, the cells preferably again exhibit normal morphology. Optimal concentration ranges can be estimated based on the above-described conditions. Evaluation of the effect in vivo can be done at several concentrations, the first of which is targeted at fine-tuning optimal concentrations and confirming cytostatic properties. Histological studies following acute and chronic exposure of the test agent is typically carried out. Certain antiproliferative agents disclosed herein have been shown to have acceptable cell compatibility in vitro, and at concentrations of from 1 to 10 mg/ml have evidenced antiproliferative effect on endothelial cell lines (e.g., human umbilical vein endothelial cells; HUVEC) and Glioblastoma/Astrocytoma cell lines (e.g., human Glioblastoma/Astrocytoma cell line U87). Interestingly at these concentrations the cell morphology appeared uncompromised, thus suggesting a cytostoatic effect.

Commercially available kits for evaluating cell growth or proliferation, cell metabolism, leakage of enzymes, and cytotoxicity can be used to evaluate the effect of test agents (e.g., cytostatic and/or cytotoxic responses).

Methods as disclosed herein can be used with a wide variety of cell lines including, but not limited to, fibroblast cells, smooth muscle cells, tumor cells, endothelial cells, and the like, and combinations thereof.

Preferred cell lines include, but are not limited to, normal human dermal fibroblast (NHDF) cells, coronary artery smooth muscle cells (CASMC), human Glioblastoma/Astrocytoma U87 cells, human umbilical vein endothelial cell (HUVEC), human coronary artery endothelial cells (HCAEC), and combinations thereof.

Methods as disclosed herein can be used to treat or prevent a wide variety of proliferative (e.g. hyperproliferative) conditions and/or diseases. Examples of such conditions and/or diseases include, but are not limited to, hyperplasia of soft and hard tissues, malignant tumors such as cancer, skin keloids, fibrosis, and surgical adhesions. Methods as disclosed herein can also be used, for example, to prevent cell proliferative processes resulting in in-stent restenosis (e.g., associated with coronary stents), pannus overgrowth on prosthetic heart valves (e.g., associated with sewing rings), urethral stenosis (e.g., associated with prostatic hyperplasia), and stenosis associated with surgical anastomosis. Methods as disclosed herein can also be used to limit tumor growth. Methods as disclosed herein can also be used to prevent the occlusion of catheters, such as, for example, cerebrospinal fluid (CSF) shunts. Methods as disclosed herein can be used, for example, to inhibit any area of the body as desired to retard excessive cell proliferation, including, for example, treatment of male sterility, inhibition of moles, prevention of excessive hair growth, and the like.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Incorporating 5,6-dihydroxyhexan-2-one into a Polymer Chain

Into a 250 mL round bottom flask, 5,6-epoxy-hexan-2-one (10.75 gram, 0.0943 mole) was dissolved in toluene (100 mL), then water (1.70 gram, 0.0943 mole) was added and stirred magnetically overnight, during which a complete homogenous solution was achieved. Without further purification, 3,9-di(ethylidene)-2,4,8,10-tetraoxaspiro[5,5]undecane (20.00 gram, 0.0943 mole) was added into the flask. 1 mg of p-toluene sulfonic acid was added after the solids went into solution. The reaction mixture was stirred magnetically overnight and then quenched into petroleum ether. Decantation and drying in a vacuum oven yielded a colorless solid. (Mn=5400 g/mol, PDI=1.9).

### Example 2

### Derivatizing 5,6-dihydroxyhexan-2-one

5,6-dihydroxyhexan-2-one was indirectly incorporated into a small molecule. In a 100 mL round bottom flask, 5,6-epoxy-hexan-2-one (1.5 grams) was dissolved in acetone (50 mL). p-Toluenesulfonic acid (PTSA; 100 micro liter of 1% pTSA in THF) was added, and an exothermic reaction occurred. After the temperature cooled to room temperature, 2 drops of triethylamine was added, and then the mixture was diluted with 15 mL saturated aqueous sodium hydrogen carbonate solution, concentrated via rotary evaporation, extracted with ether. The ether layer was subsequently washed with brine, dried with anhydrous sodium sulfate, and concentrated to give 1.43 grams product. ¹H-NMR and ¹³C-NMR confirmed the structure as the acetonide of 5,6-dihydroxy-hexan-2-one. ¹³C-NMR (CDCl₃, as solvent and reference at 77 ppm), 25.64, 26.95, 27.44, 30.00, 69.23, 75.06,108.95, 208.07. ¹H-NMR (CDCl₃, tetramethylsilane (TMS) as reference at 0.0 ppm) 1.23 (s, 3H), 1.34 (s, 3H), 1.72 (doublet of multiplets, 2H), 2.11 (s, 3H), 2.51 (m, 2H), 2.47 (d, 1H), 3.96 (d, 1H), 4.00 (m, 1H).

### Example 3

Examination of the Cytotoxic Effects Upon 24 Hour and 48 Hour Exposure of Normal Human Dermal Fibroblast (NHDF) Cells to 5,6-dihydroxy-hexan-2-one

The assay used was a non-radioactive cytotoxicity assay available under the trade designation CytoTox 96 from Promega Corporation (Madison, Wis.), which was designed to determine the cytotoxicity of a substance by quantitatively measuring the presence of lactate dehydrogenase (LDH) in cell culture supernatant. The enzymatic assay utilizes the LDH released in the supernatant in the conversion of a tetrazolium salt substrate into a red formazan product. This resulting product is measured using a standard 96 well plate reader where the absorbance recorded at 490 nanometers is directly proportional to the number of lysed cells. The Normal Human Dermal Fibroblasts were available from Clonetics Corporation (San Diego, Calif.).

The 5,6-dihydroxy-hexan-2-one (1.2 grams) was dissolved in 12 ml of phosphate buffered saline and was filter sterilized through a 0.22 micrometer syringe filter prior to use. The 5,6-dihydroxy-hexan-2-one was further diluted to 10⁻¹, 10⁻², 10⁻³, 10⁻⁴, 10⁻⁵, and 10⁻⁶ using serial dilutions for the evaluation.

NHDF were seeded at 45,000 cells/well in 24 well plate(s). Cells were grown for 72 hours, 48 hours of which was done in the presence of the test chemicals. 24 hours after seeding, 5,6-dihydroxy-hexan-2-one was added to the cell culture plates (2 ml/well). Assay plates were pulled for analysis at 24 and 48 hours after addition of the chemical compound. Samples were analyzed on the above-described assay as provided in the instructions for use of the assay.

Cell counts were taken after the cells had been plated for 72 hours and exposed to 5,6-dihydroxy-hexan-2-one for 48 hours.

FIG. 1 is a graphical representation of the cytotoxicity of fibroblast cells exposed to various concentrations of 5,6-dihydroxy-hexan-2-one for 24 hours and 48 hours. Negligible levels (from 0 to 2.5% on a scale of 0 to 100%) were observed for controls and test groups. The results illustrated in FIG. 1 indicate no increase in cytotoxicity as a result of exposing the cells to the various concentrations of 5,6-dihydroxy-hexan-2-one for 48 hours.

The cytotoxic effect of 5,6-dihydroxy-hexan-2-one was qualitatively analyzed by a visual morphological study and quantitatively by the above described assay following cell exposure to 5,6-dihydroxy-hexan-2-one for 24 and 48 hours.

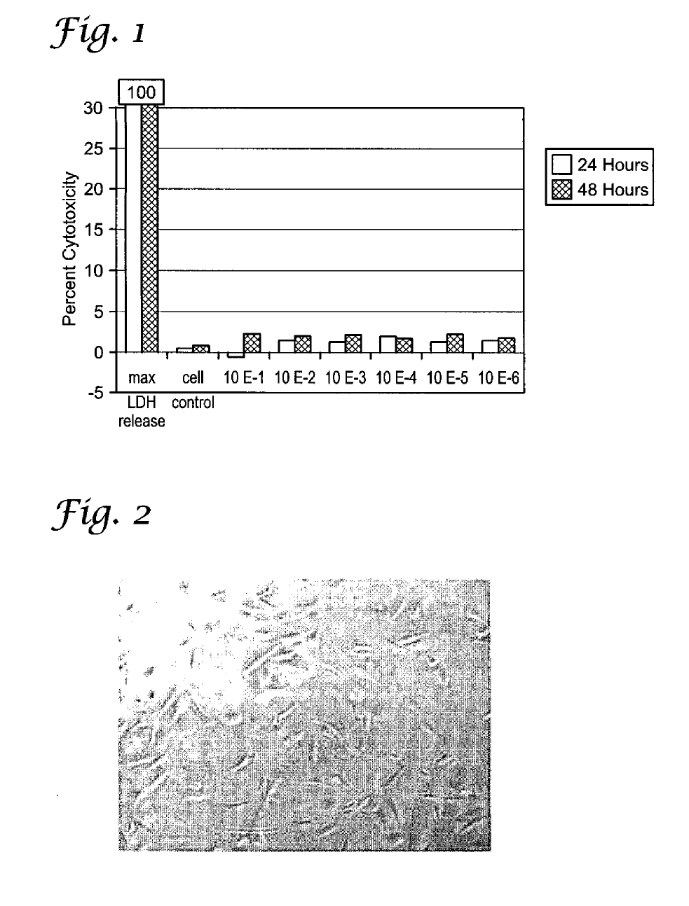

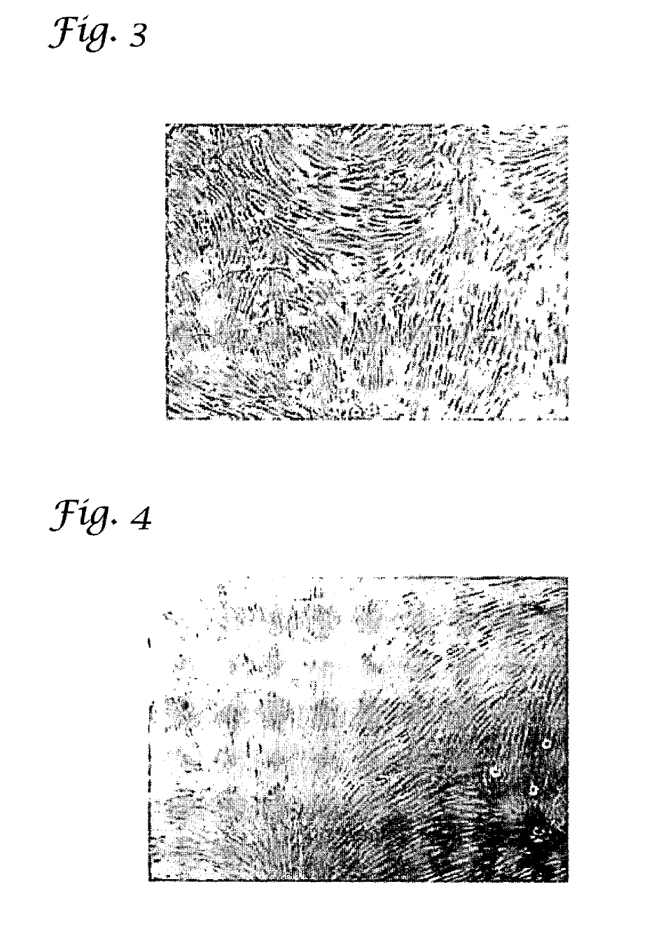

FIG. 2 (10⁻¹, 48 hours), FIG. 3 (10⁻³, 48 hours), and FIG. 4 (10⁻⁶, 48 hours) visually display the physical cellular response of NHDF to various concentrations of 5,6-dihydroxy-hexan-2-one. The most notable physical changes to the NHDF occurred in the 10⁻¹ concentration of 5,6-dihydroxy-hexan-2-one dissolved in fibroblast specific medium. Fewer human dermal fibroblasts cells were observed as a result of exposure to the high concentration of 5,6-dihydroxy-hexan-2-one. Lower concentrations of 5,6-dihydroxy-hexan-2-one did not elicit such an observation.

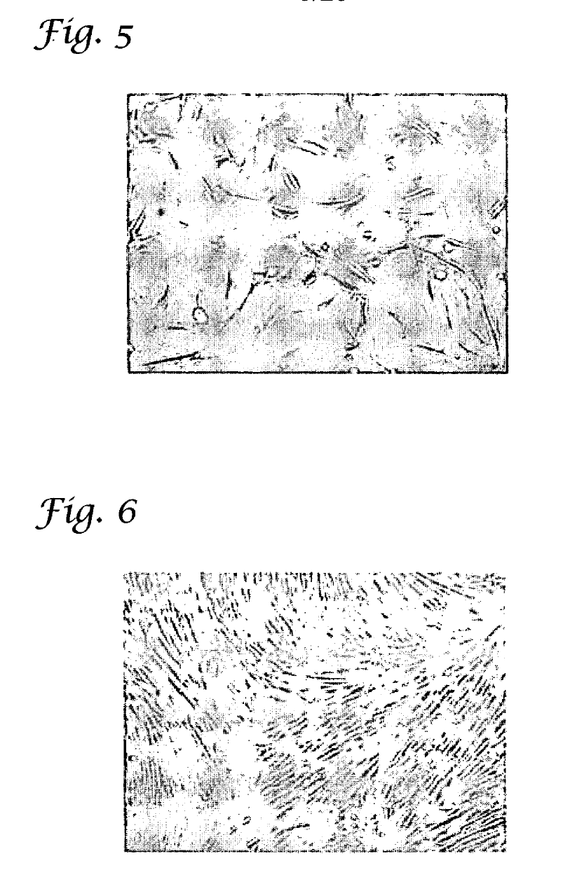

After 48 hours of exposure to 5,6-dihydroxy-hexan-2-one, a cell count was taken on the 10⁻¹ concentration group and the 10⁻⁶ concentration group. The 10⁻¹ cell count was 45,000 cells/well and the 10⁻⁶ cell count was 275,000 cells/well. The count for the 10⁻¹ concentration group yielded the same number of cells that were previously seeded, while the 10⁻⁶ concentration group had continued to grow rapidly without problems. Cells from these two different groups were then plated out at the same density as before, but without exposure to 5,6-dihydroxy-hexan-2-one and in fresh media for 96 hours. Visual representations of these two groups can be seen in FIG. 5 (10⁻¹, exposed to 5,6-dihydroxy-hexan-2-one for 48 hours, continued the culture for an additional 96 hours without 5,6-dihydroxy-hexan-2-one) and FIG. 6 (10⁻⁶, exposed to 5,6-dihydroxy-hexan-2-one for 48 hours, continued the culture for an additional 96 hours without 5,6-dihydroxy-hexan-2-one). The cells in the 10⁻⁶ concentration group continued to grow without problems, while the cells in the 10⁻¹ group appeared to have an impaired ability for cell proliferation (growth).

The cytotoxicity assay revealed no significant cytotoxicity in response to the presence of 5,6-dihydroxy-hexan-2-one, at 10⁻¹ concentration, indicating minimal release of LDH attributable to cell death. However the ability for cells to proliferate in the presence of 5,6-dihydroxy-hexan-2-one was significantly affected. Furthermore, the culture of previously exposed cells in a media without 5,6-dihydroxy-hexan-2-one indicated a persistent negative effect on proliferation. This may suggest accumulative anti-proliferative effect or the possibility of agent remaining in the in vitro system.

In summary, the visual results suggest that 5,6-dihydroxy-hexan-2-one at a concentration of 10⁻¹ for NHDF elicit signs of cell distress at 24 and 48 hours in vitro, a response not observed with lower concentrations of 5,6-dihydroxy-hexan-2-one. Although, a general, low cytotoxicity was observed (LDH release), the highest concentration condition elicited a significant and persistent impairment of the cell proliferation indicating a dose-effect phenomena. The apparent absence of cytotoxicity observed for lower concentrations of 5,6-dihydroxy-hexan-2-one in this in vitro setting may not necessarily be directly correlated with a given material's chronic biocompatibility or lack of cytotoxicity in vivo. The actual in vivo tissue-biomaterial conditions, dosages, accumulation/clearance, and durations for agent exposure to cells/tissue may vary according to the specific application and/or material formulations.

### Example 4

Examination of the cell growth effects upon 24 hour and 48 hour exposure of Normal Human Dermal Fibroblasts (NHDF) to 5,6-dihydroxy-hexan-2-one. After the 24 hour or 48 hour exposures, the 5,6-dihydroxy-hexan-2-one was removed and the cells were allowed to grow for an additional 96 hours, then evaluated for growth.

The assay used was a luminescent cell viability assay available under the trade designation CellTiter-Glo from Promega Corporation (Madison, Wis.). This assay determines the number of viable cells present based on the amount of adenosine triphosphate (ATP) present from the cell cytoplasm. The concentration of ATP present is proportional to the measured amount of a luminescent signal. The amount of ATP shows the existence of metabolically active cells, and hence, the number of viable cells. The Normal Human Dermal Fibroblasts were available from Clonetics Corporation (San Diego, Calif.).

The 5,6-dihydroxy-hexan-2-one (1.2 grams) was dissolved in 12 ml of phosphate buffered saline and was filter sterilized through a 0.22 micrometer syringe filter prior to use. The 5,6-dihydroxy-hexan-2-one was further diluted to 10⁻¹, 10⁻², and 10⁻³ using serial dilutions for the evaluation.

NHDF were seeded at 45,000 cells/well in four 24 well plates (two testing cell growth and two to re-seed after 24 and 48 hours without 5,6-dihydroxy-hexan-2-one). 24 hours after seeding, 5,6-dihydroxy-hexan-2-one was added to the cell culture plates (2 ml/well) at a concentration of 10⁻¹, 10⁻², and 10⁻³. Assay plates were pulled for cell growth analysis at 24 and 48 hours after addition of the chemical compound. Samples were analyzed on the above-described assay according to a published method. The other two assay plates were washed and re-seeded at 45,000 cells/well at 24 and 48 hour time points without the presence of 5,6-dihydroxy-hexan-2-one. Both plates were grown for an additional 96 hours before a cell count was taken.

Cell counts taken after the cells had been plated for 72 hours and exposed to 5,6-dihydroxy-hexan-2-one for 24 hours and 48 hours are shown in Table 1.

TABLE 1
Cell Counts after exposure to 5,6-dihydroxy-hexan-2-one
for 24 hours and 48 hours. Control cells were not exposed to 5,6-
dihydroxy-hexan-2-one.

5,6-Dihydroxy-hexan-2-one
Concentration

| | | | | Cell |
|---|---|---|---|---|
| | 10 mg/ml | 1 mg/ml | 0.1 mg/ml | Control |
| 24 Hour Group | 65,000 | 160,000 | 180,000 | 260,000 |
| | cells | cells | cells | cells |
| 48 Hour Group | 80,000 | 150,000 | 305,000 | 210,000 |
| | cells | cells | cells | cells |

In addition, cell counts were taken after the cells had been plated for 72 hours, exposed to 5,6-dihydroxy-hexan-2-one for 24 hours and 48 hours, harvested, and then re-seeded without 5,6-dihydroxy-hexan-2-one. The cells were allowed to grow in fresh media for an additional 96 hours. The cell counts are shown in Table 2.

TABLE 2
Cell Counts after exposure to 5,6-dihydroxy-hexan-2-one
for 24 hours and 48 hours and allowed to grow for an additional
96 hours with the 5,6-dihydroxy-hexan-2-one removed. Control
cells were not exposed to 5,6-dihydroxy-hexan-2-one.

5,6-Dihydroxy-hexan-2-one
Concentration

| | | | | Cell |
|---|---|---|---|---|
| | 10 mg/ml | 1 mg/ml | 0.1 mg/ml | Control |
| 24 Hour Group | 55,000 | 172,000 | 200,000 | 200,000 |
| | cells | cells | cells | cells |
| 48 Hour Group | 65,000 | 165,000 | 207,500 | 237,000 |
| | cells | cells | cells | cells |

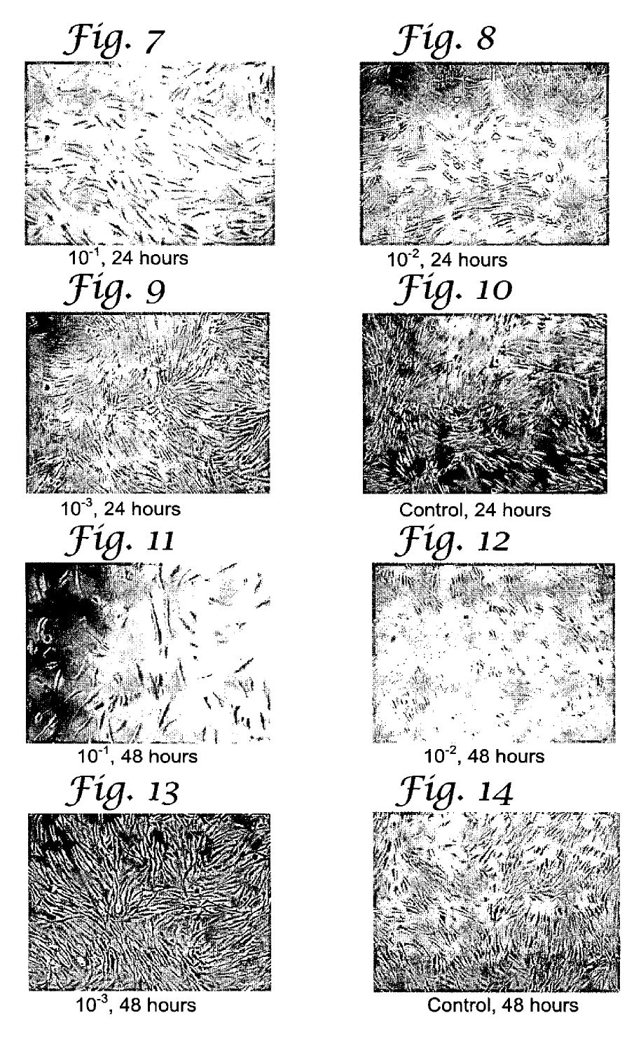

FIG. 7 (10⁻¹, 24 hours), FIG. 8 (10⁻², 24 hours), FIG. 9 (10⁻³, 24 hours), FIG. 10 (control, 24 hours), FIG. 11 (10⁻¹, 48 hours), FIG. 12 (10⁻², 48 hours), FIG. 13 (10⁻³, 48 hours), and FIG. 14 (control, 48 hours) visually display the physical cellular response of NHDF to various concentrations of 5,6-dihydroxy-hexan-2-one. The most notable physical changes to the NHDF occurred in the highest concentration (10⁻¹) of 5,6-dihydroxy-hexan-2-one dissolved in fibroblast specific medium. Fewer human dermal fibroblasts cells were observed as a result of exposure to 5,6-dihydroxy-hexan-2-one. Lower concentrations did not elicit such a response.

After 24 and 48 hours of exposure to 5,6-dihdroxy-hexan-2-one, cell counts were taken on all three different concentration groups (Table 1). The count for the 10⁻¹ concentration group yielded only a minimal amount of growth, while the 10⁻² had additional cell proliferation, but still reduced from the control. The 10⁻³ concentration group continued to grow without problems when compared to the cell control.

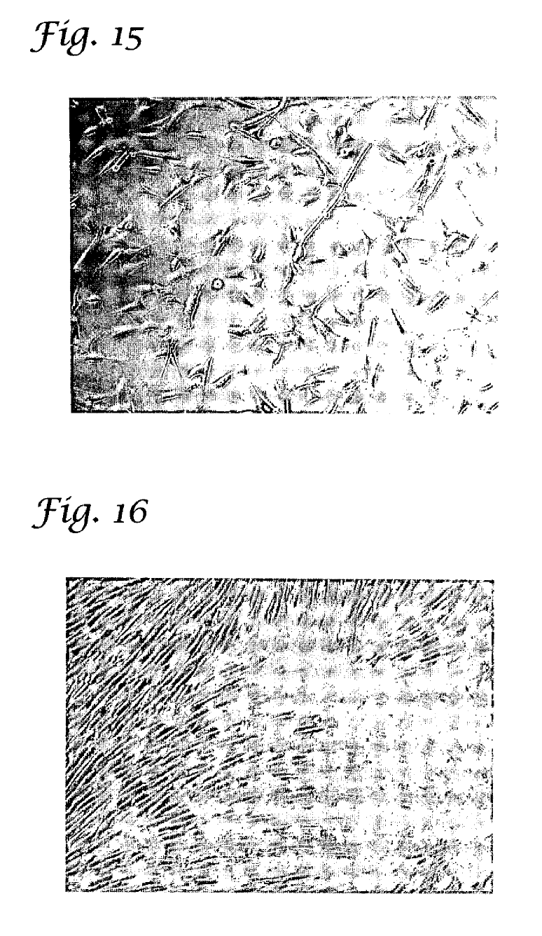

To determine proliferation potential post-exposure, cells were reseeded after exposure to 5,6-dihydroxy-hexan-2-one as follows. Additional groups of cells exposed to 5,6-dihydroxy-hexan-2-one for 24 and 48 hours were then washed thoroughly to remove 5,6-dihydroxy-hexan-2-one and harvested from the culture substrate. Those cells were then plated out at the same density as before, but without exposure to 5,6-dihydroxy-hexan-2-one. These two groups were supplied with fresh media and allowed to grow for 96 additional hours. Visual representation of the 10⁻¹ and 10⁻² groups can be seen in FIG. 15 (10⁻¹, re-seeded without 5,6-dihydroxy-hexan-2-one for 96 hours) and FIG. 16 (10⁻², re-seeded without 5,6-dihydroxy-hexan-2-one for 96 hours). The cells in the 10⁻² concentration group continued to grow without problems, while the cells in the 10⁻¹ group appeared to have a slower growth.

Table 2 represents a cell count taken of the different concentration groups after 96 hours of growing without the presence of 5,6-dihydroxy-hexan-2-one. Although, a slight reduction of cell growth was observed in the 10⁻² group, the cell count matched what was visually seen, as did the 10⁻¹ and 10⁻³ concentrations. The 10⁻¹ group showed significant inhibition of cell growth and had roughly the same amount of cells that were previously seeded. The cells in the 10⁻¹ group have proven to have an impaired ability for cell proliferation or growth.

These in vitro studies suggest a direct dose-effect of 5,6-dihydroxy-hexan-2-one on cell proliferation. Thus the absolute total cell number is significantly decreased during exposure to 5,6-dihydroxy-hexan-2-one. Likewise, the agent's effect on cells subsequent to the exposure yielded interesting observations in which dose was directly correlated to cells ability to regain their ability for proliferation. Cells incubated in the presence of 10⁻³ concentration (the lowest concentration tested) appear to behave similar to controls (w/o agent) soon after the 5,6-dihydroxy-hexan-2-one is withdrawn. In contrast, cells incubated in 10⁻² and particularly 10⁻¹ concentrations showed an impaired ability to regain their proliferative potential following withdrawal of the 5,6-dihydroxy-hexan-2-one.

### Example 5

Evaluation of the effects of 5,6-dihydroxy-hexan-2-one on cell proliferation, viability, and migration of normal human dermal fibroblast (NHDF) cells and coronary artery smooth muscle cells (CASMC) performed in vitro.

Multiple assays were used to test a variety of cellular properties expressed after exposure of different cells types to 5,6-dihydroxy-hexan-2-one. A cell proliferation assay available under the trade designation CyQuant from Invitrogen Corporation (Carlsbad, Calif.) determines the density of cells in culture by using a green fluorescent dye which fluoresces when bound to cellular nucleic acids. The amount of nucleic acid present is proportional to the amount of living cells in culture. A luminescent cell viability assay available under the trade designation CellTiter-Glo from Promega Corporation (Madison, Wis.) uses the amount of ATP present in the cell cytoplasm as an indicator for metabolic activity.

The amount of ATP present is proportional to the measured amount of a luminescent signal and therefore the amount of viable cells. An aqueous one solution cell proliferation assay available under the trade designation CellTiter 96 from Promega Corporation (Madison, Wis.) determines the amount of NADPH or NADH produced by dehydrogense enzymes in metabolically active cells. The absorbance read is proportional to the amount of metabolic activity present in the cells.

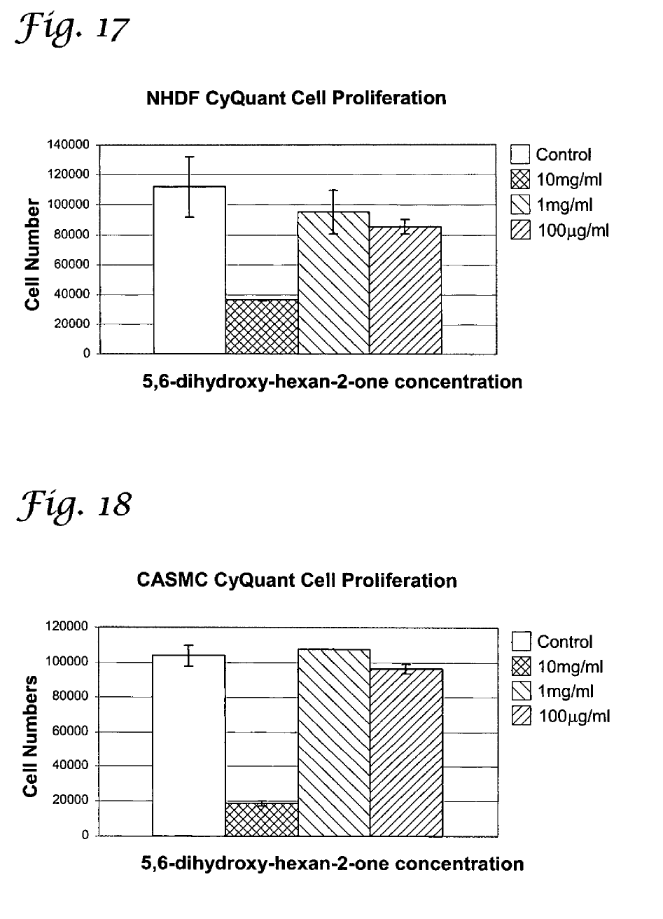

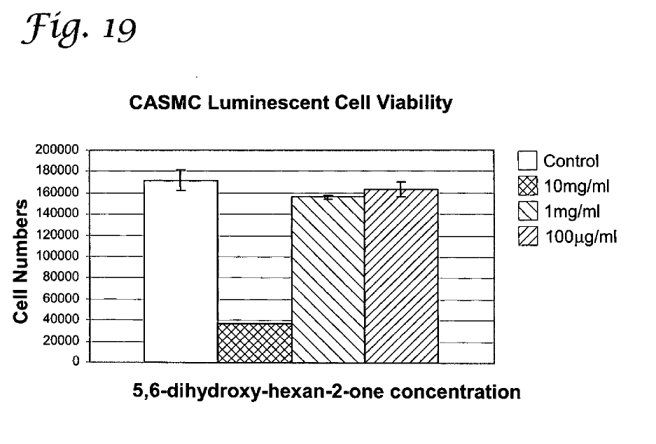

Normal human dermal fibroblast (NHDF) cells and coronary artery smooth muscle cells (CASMC) in culture were exposed to 5,6-dihydroxy-hexan-2-one at concentrations of 10 microgram/ml, 1 mg/ml, and 10 mg/ml, for 48 hours, after which the cell culture wells were analyzed for cell proliferation and viability. Controls cells were not exposed to 5,6-dihydroxy-hexan-2-one. FIG. 17 is a graphical representation of fibroblast cell numbers measured after 48 hours exposure to different concentrations of 5,6-dihydroxy-hexan-2-one. FIG. 18 is a graphical representation of smooth muscle cell numbers measured after 48 hours exposure to different concentrations of 5,6-dihydroxy-hexan-2-one. FIG. 19 is a graphical representation of viable smooth muscle cell numbers measured after 48 hours exposure to different concentrations of 5,6-dihydroxy-hexan-2-one.

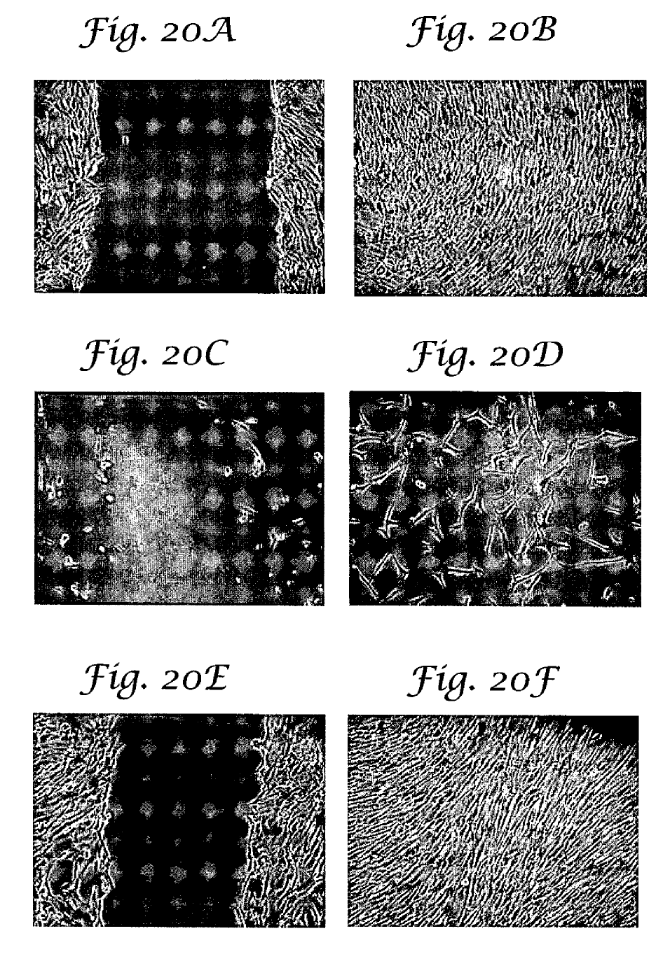

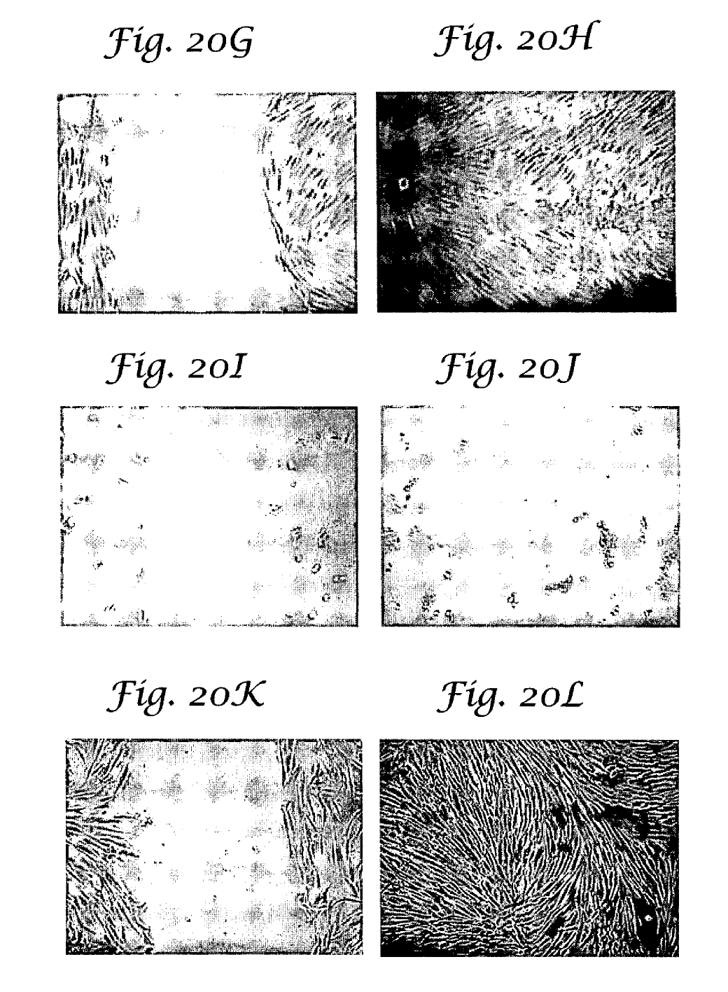

In a similar manner, cell migration was explored in vitro by scraping the cell monolayer and culturing the cells in the presence of 5,6-dihydroxy-hexan-2-one at concentrations of 1 mg/ml and 10 mg/ml. FIGS. 20(a)-(l) are pictures that visually display the migration of coronary artery smooth muscle cells (CASMC) (FIGS. 20(a)-(f)) and normal human dermal fibroblast (NHDF) cells (FIGS. 20(g)-(l)) exposed to various concentrations of 5,6-dihydroxy-hexan-2-one after 48 hours exposure, then cultured an additional 72 hours after removal of the 5,6-dihydroxy-hexan-2-one as described in Example 5. Specifically, FIGS. 20(a) and (b) represent the CASMC control at time zero and 72 hours, respectively; FIGS. 20(c) and (d) represent CASMC (10 mg/ml) at time zero and 72 hours, respectively; and FIGS. 20(e) and (f) represent CASMC (1 mg/ml) at time zero and 72 hours, respectively. FIGS. 20(g) and (h) represent the NHDF cell control at time zero and 72 hours, respectively; FIGS. 20(i) and (j) represent NHDF cells (10 mg/ml) at time zero and 72 hours, respectively; and FIGS. 20(k) and (l) represent NHDF cells (1 mg/ml) at time zero and 72 hours, respectively.

The results indicated inhibition of cell proliferation of NHDF and CASMC at 10 mg/ml 5,6-dihydroxy-hexan-2-one concentration. In contrast, other concentrations of 5,6-dihydroxy-hexan-2-one did not show this effect.

Cell migration judged by the ability of cells to repopulate the scraped “acellular” space over time indicated no microscopically identifiable effect by the agent at the concentrations tested. Thus, cells treated with the agent for 48 hours and allowed to grow for 72 hours were able to repopulate the scraped spaces in all conditions.

In summary, the assays and visual observations suggested that 5,6-dihydroxy-hexan-2-one at a concentration of 10 mg/ml has an antiproliferative effect, and that cell migration as evaluated in this example was not affected by treatment with 5,6-dihydroxy-hexan-2-one at 1 and 10 mg/ml concentrations.

### Example 6

Evaluation of the effects of 5,6-dihydroxy-hexan-2-one on human umbilical vein endothelial cells (HUVEC) and Glioblastoma/Astrocytoma cells U87 cellular proliferation and migration during 120 hours following 48 hour exposure to 5,6-dihydroxy-hexan-2-one at concentration of 10 mg/ml, 1 mg/ml, 100 micrograms/ml. Controls were cells not exposed to the agent.

Preliminary evaluation had suggested an antiproliferative effect induced by the presence of 5,6-dihydroxy-hexan-2-one in cell culture preparations. In order to further explore the effects of this compound, experiments to evaluate the dose-effect response on the antiproliferative effect and on cell migration were conducted on two cell lines, human umbilical vein endothelial cells (HUVEC) and Glioblastoma/Astrocytoma cells (U87), over 120 hours after 48 hours of exposure to the compound.

A cell proliferation assay available under the trade designation CyQuant from Invitrogen Corporation (Carlsbad, Calif.) determines the density of cells in culture by using a green fluorescent dye which fluoresces when bound to cellular nucleic acids.

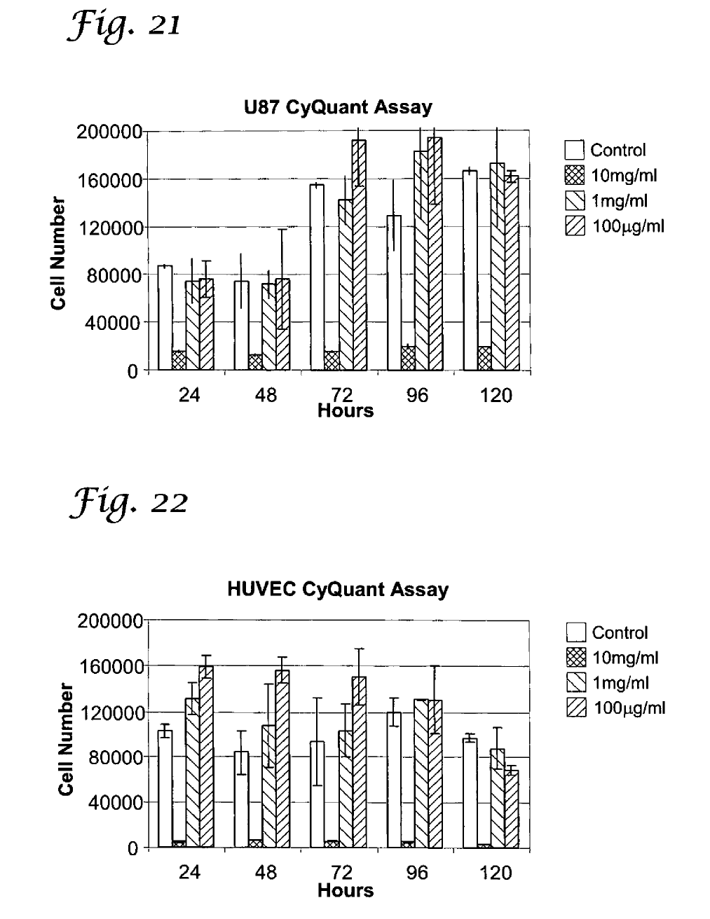

FIG. 21 is a graphical representation of human Glioblastoma/Astrocytoma U87 cell numbers measured after being exposed to various concentrations of 5,6-dihydroxy-hexan-2-one for 48 hours. Time 0 refers to the replacement of the test agent with supplemented growth media.

FIG. 22 is a graphical representation of human umbilical vein endothelial cell (HUVEC) numbers measured after being exposed to various concentrations of 5,6-dihydroxy-hexan-2-one for 48 hours. Time 0 refers to replacement of the test agent with supplemented growth media.

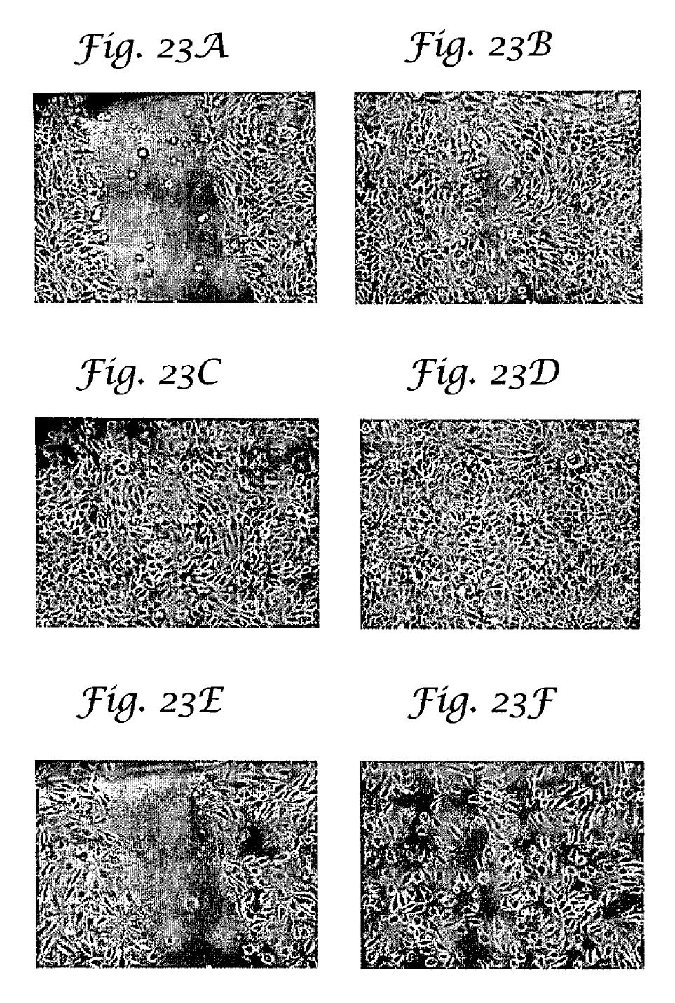

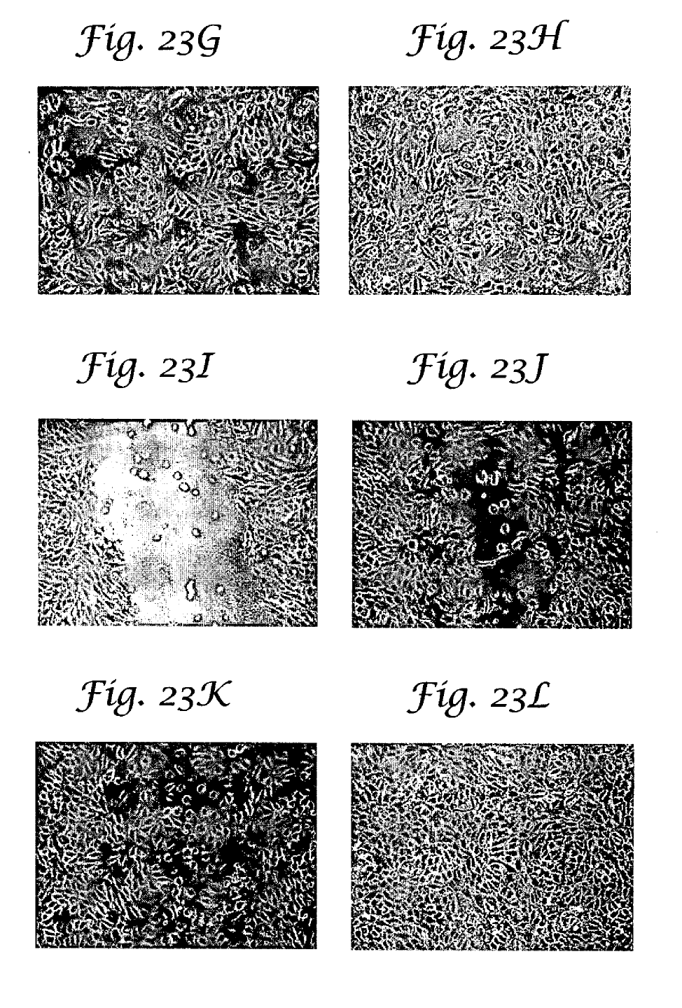

FIGS. 23(a)-(l) are pictures that visually display the migration over time of human umbilical vein endothelial cells (HUVEC) after 48 hours exposure to various concentrations of 5,6-dihydroxy-hexan-2-one. Specifically, FIGS. 23(a)-(d) represent the control, FIGS. 23(e)-(h) represent 10 mg/ml, and FIGS. 23(i)-(l) represent 1 mg/ml 5,6-dihydroxy-hexan-2-one. FIGS. 23(a), (e), and (i) represent migration after 2 hours; FIGS. 23(b), (f), and (j) represent migration after 14 hours; FIGS. 23(c), (g), and (k) represent migration after 20 hours; and FIGS. 23(d), (h), and (l) represent migration after 48 hours.

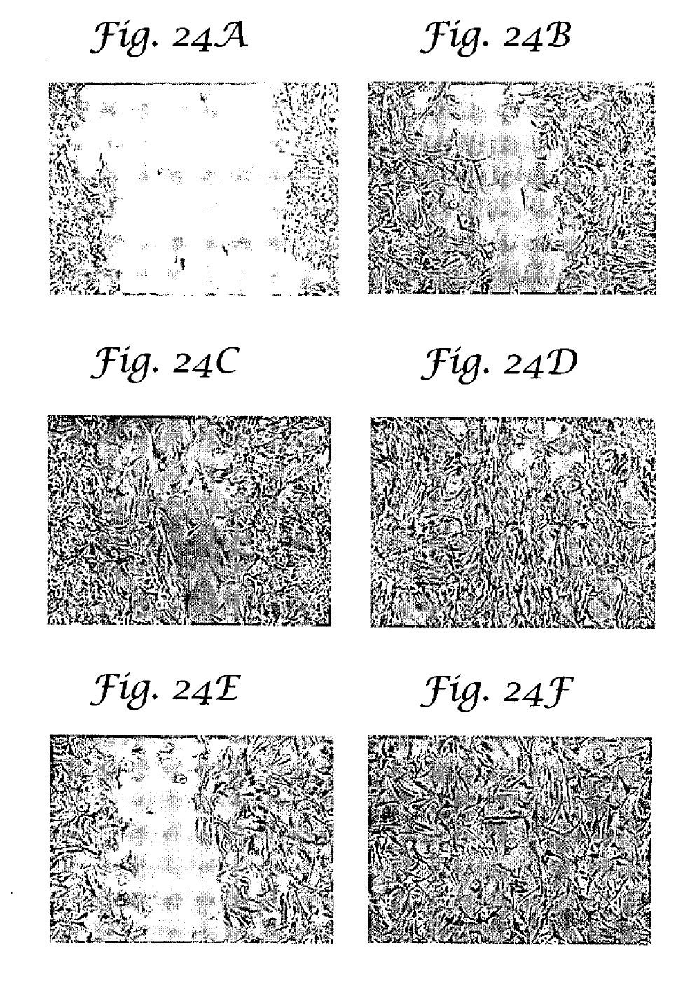

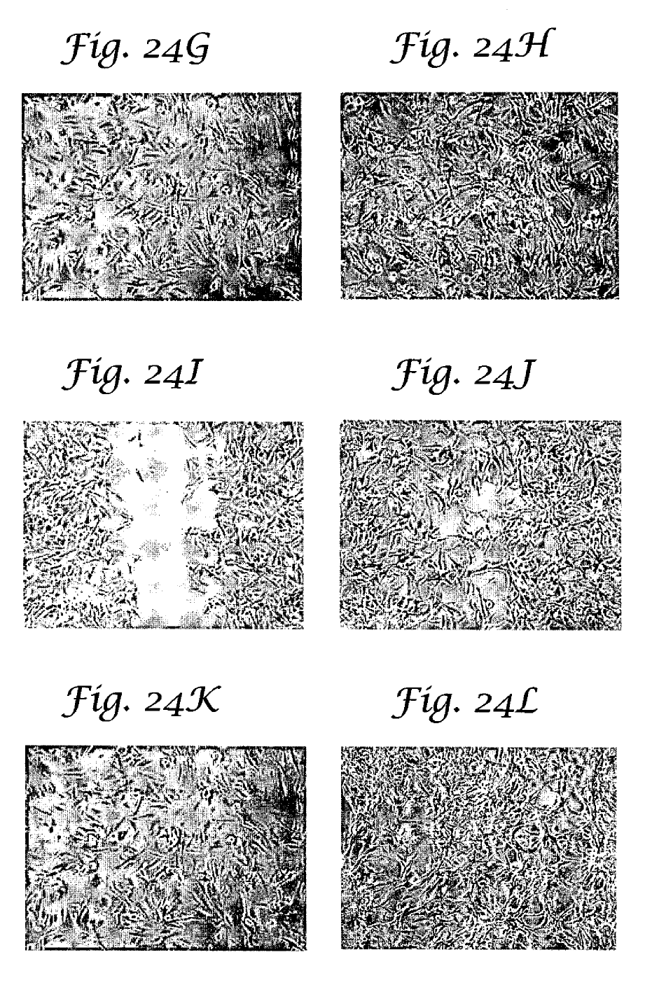

FIGS. 24(a)-(l) are pictures that visually display the migration over time of human Glioblastoma/Astrocytoma U87 cells after 48 hours exposure to various concentrations of 5,6-dihydroxy-hexan-2-one. Specifically, FIGS. 24(a)-(d) represent the control, FIGS. 24(e)-(h) represent 10 mg/ml, and FIGS. 24(i)-(l) represent 1 mg/ml 5,6-dihydroxy-hexan-2-one. FIGS. 24(a), (e), and (i) represent migration after 2 hours; FIGS. 24(b), (f), and (j) represent migration after 14 hours; FIGS. 24(c), (g), and (k) represent migration after 20 hours; and FIGS. 23(d), (h), and (l) represent migration after 48 hours.

The results indicated no antiproliferative effects observed at the lowest concentration evaluated. 5,6-dihydroxy-hexan-2-one at 10 mg/ml showed antiproliferative effects on the two cell lines used in this evaluation. This inhibitory effect was observed as late as 120 hours following the withdrawal of the 5,6-dihydroxy-hexan-2-one from cell cultures. Experiments on cell migration did not shown differences between control and test conditions, thus by 20 hours, the cells migrated and filled the scraped space (i.e., as the assay indicated inhibition of cell proliferation).

### Example 7

Evaluation of the Effects Upon 24 and 48 Hour Exposure of Human Coronary Artery Endothelial Cells (HCAEC) to Six Compounds

The compounds in Table 3 were evaluated.

TABLE 3
Compounds Evaluated

| Designation | Structure | Name |
|---|---|---|
| 13358-18-1 | 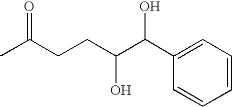 | 5,6-dihydroxy-6-phenyl-hexan-2-one |
| 13358-18-2 | 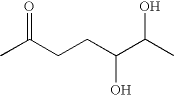 | 5,6-dihydroxy-heptan-2-one |
| 13358-18-3 | 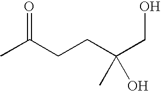 | 5,6-dihydroxy-5-methyl-hexan-2-one |
| 13358-18-4 | 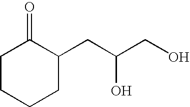 | 2-(2′,3′-dihydroxypropyl)-cyclohexanone |
| 13358-18-5 | 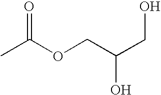 | 2,3-dihydroxy-propyl acetate |
| 13358-18-6 | 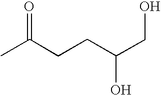 | 5,6-dihydroxy-hexan-2-one |

Cells were exposed to the test compounds for 24 to 48 hours and then allowed to grow for 96 hours in regular media (without test compound). The final concentrations for cell treatment were as follows: compounds 13358-18-1, 13358-18-2, 13358-18-4, and 13358-18-6 were evaluated at 10 mg/ml, 1 mg/ml, 100 micrograms/ml, and 10 micrograms/ml. Compounds 13358-18-3 and 13358-18-5 were evaluated at 50 mg/ml, 10 mg/ml, 1 mg/ml, and 100 micrograms/ml.

Three assays were used to look at the proliferation, viability and cytotoxicity of the compounds on the cells. A non-radioactive cytotoxicity assay available under the trade designation CytoTox 96 from Promega Corporation (Madison, Wis.) is designed to determine the cytotoxicity of a substance by quantitatively measuring the presence of lactate dehydrogenase (LDH) in cell culture supernatant. LDH is released in cells that are undergoing cell death and the assay converts it to a red formazan product that can allows for the proportional amount of LDH absorbance to be read. A cell proliferation assay available under the trade designation CyQuant from Invitrogen Corporation (Carlsbad, Calif.) determines the density of cells in culture by eluding a green fluorescent dye which fluoresces when bound to cellular nucleic acids. The amount of nucleic acid present is proportional to the amount of living cells in culture, therefore an accurate cell count can be obtained. A luminescent cell viability assay available under the trade designation CellTiter-Glo from Promega Corporation (Madison, Wis.) uses the amount ATP present in the cell cytoplasm as an indicator for metabolic activity. The amount of ATP present is proportional to the measured amount of a luminescent signal. This assay was used to confirm if the cells are still viable after exposure to the test compounds.

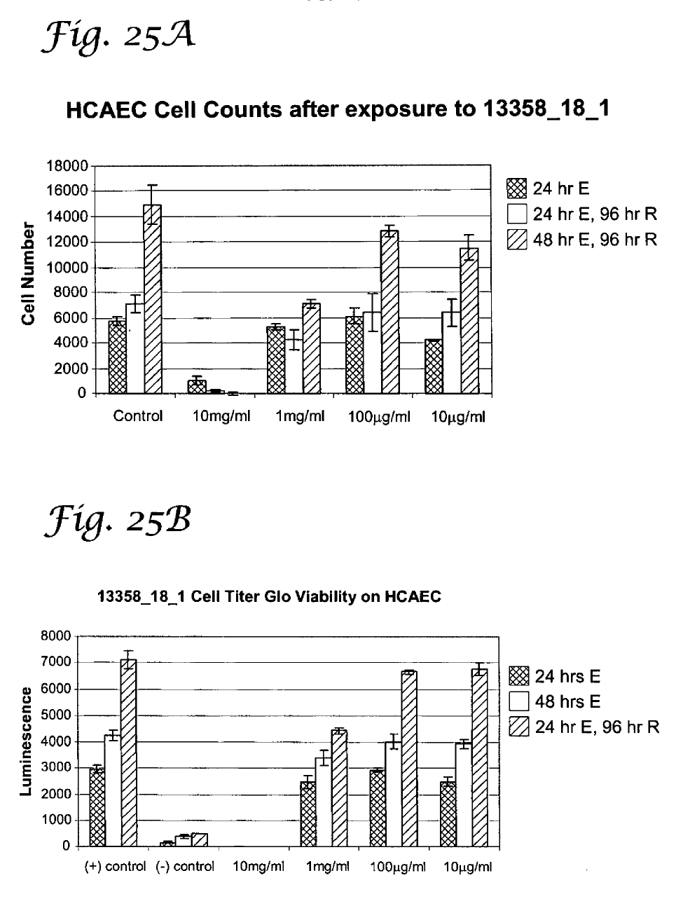

FIGS. 25(a) and (b) are graphical illustrations of cell counts and cell viability, respectively, for human coronary artery endothelial cells (HCAEC) exposed to 5,6-dihydroxy-6-phenyl-hexan-2-one (13358-18-1) for 24 hours and 48 hours, then allowed to grow for 96 hours after removal of 5,6-dihydroxy-6-phenyl-hexan-2-one.

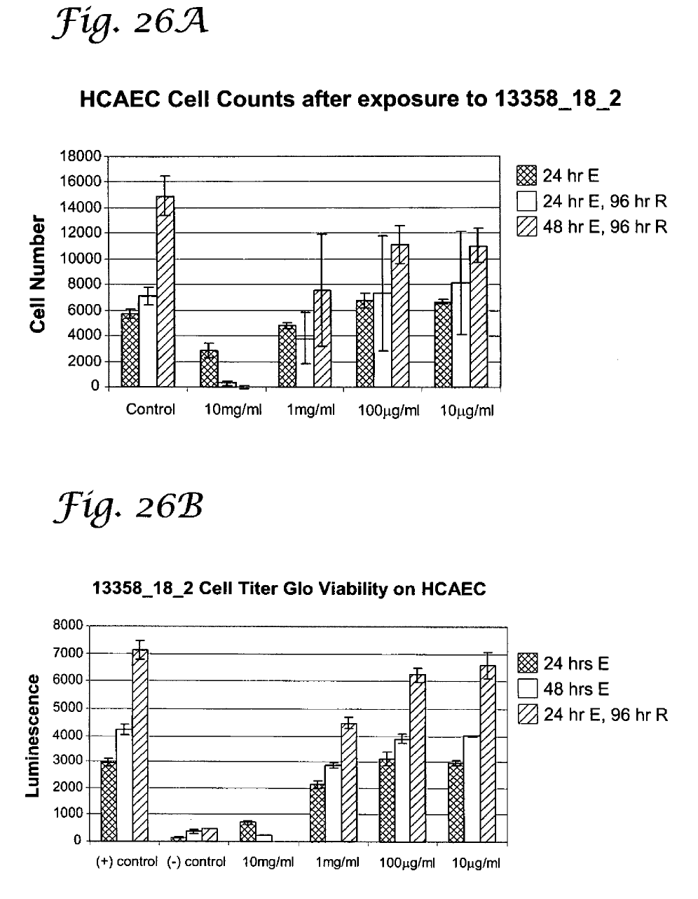

FIGS. 26(a) and (b) are graphical illustrations of cell counts and cell viability, respectively, for human coronary artery endothelial cells (HCAEC) exposed to 5,6-dihydroxy-heptan-2-one (13358-18-2) for 24 hours and 48 hours, then allowed to grow for 96 hours after removal of 5,6-dihydroxy-heptan-2-one.

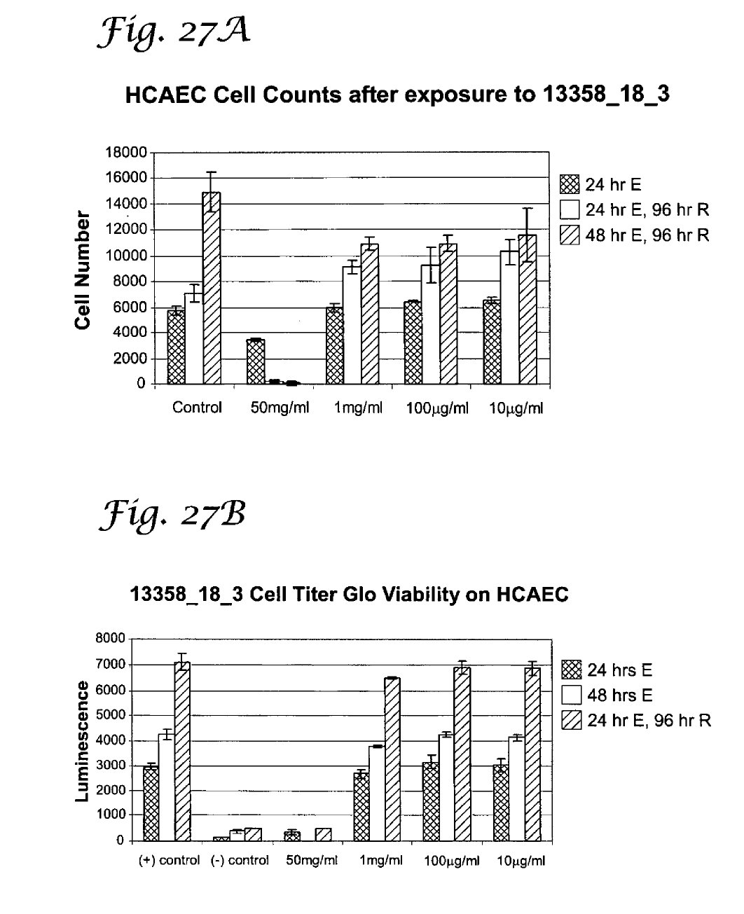

FIGS. 27(a) and (b) are graphical illustrations of cell counts and cell viability, respectively, for human coronary artery endothelial cells (HCAEC) exposed to 5,6-dihydroxy-5-methyl-hexan-2-one (13358-18-3 for 24 hours and 48 hours, then allowed to grow for 96 hours after removal of 5,6-dihydroxy-5-methyl-hexan-2-one.

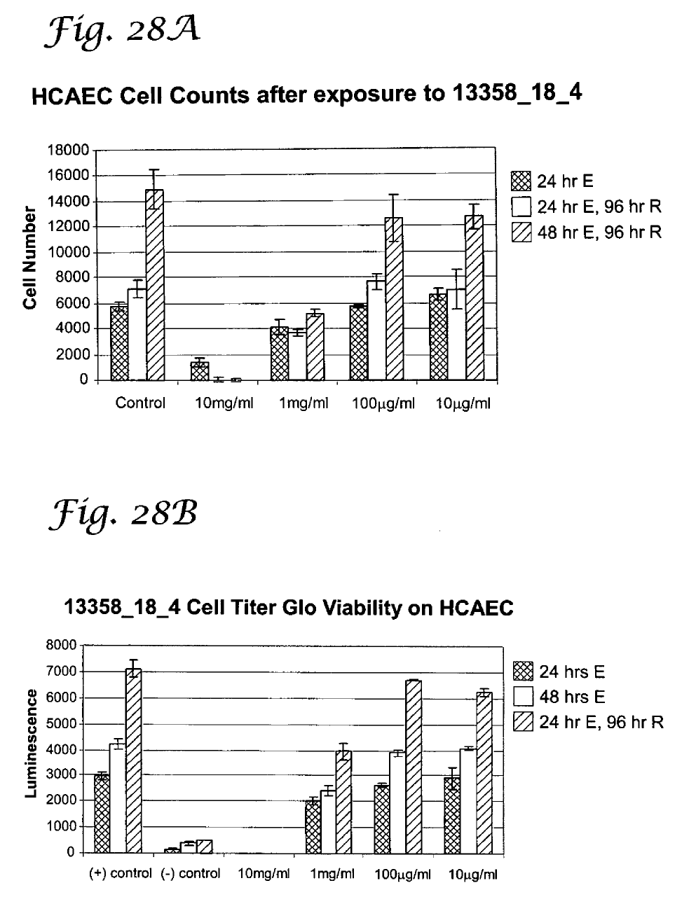

FIGS. 28(a) and (b) are graphical illustrations of cell counts and cell viability, respectively, for human coronary artery endothelial cells (HCAEC) exposed to 2-(2′,3′-dihydroxypropyl)-cyclohexanone (13358-18-4) for 24 hours and 48 hours, then allowed to grow for 96 hours after removal of 2-(2′,3′-dihydroxypropyl)-cyclohexanone.

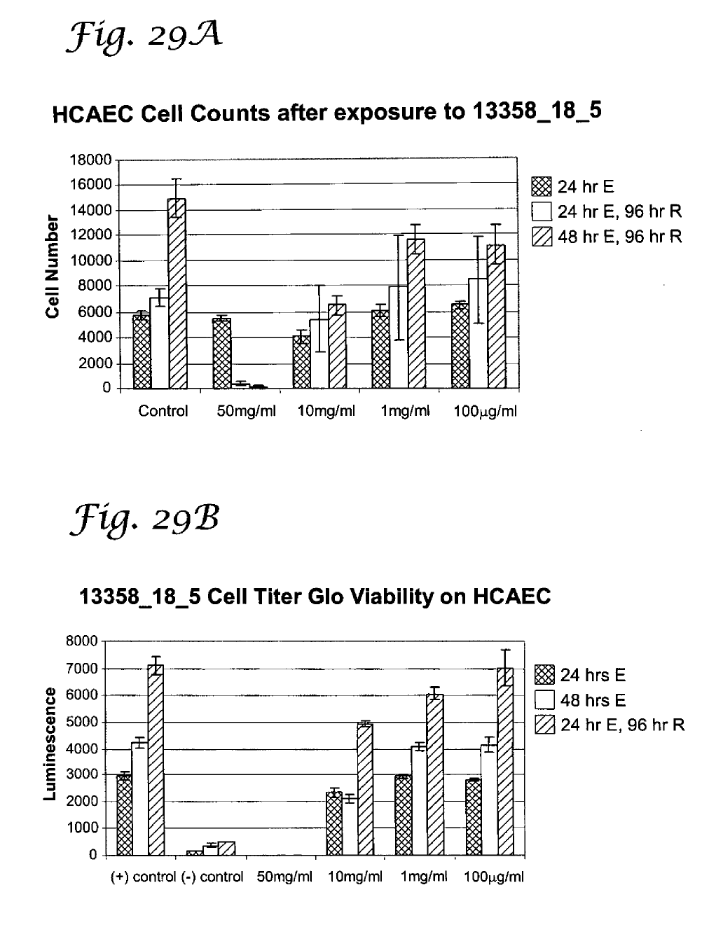

FIGS. 29(a) and (b) are graphical illustrations of cell counts and cell viability, respectively, for human coronary artery endothelial cells (HCAEC) exposed to 2,3-dihydroxypropyl acetate (13358-18-5) for 24 hours and 48 hours, then allowed to grow for 96 hours after removal of 2,3-dihydroxypropyl acetate.

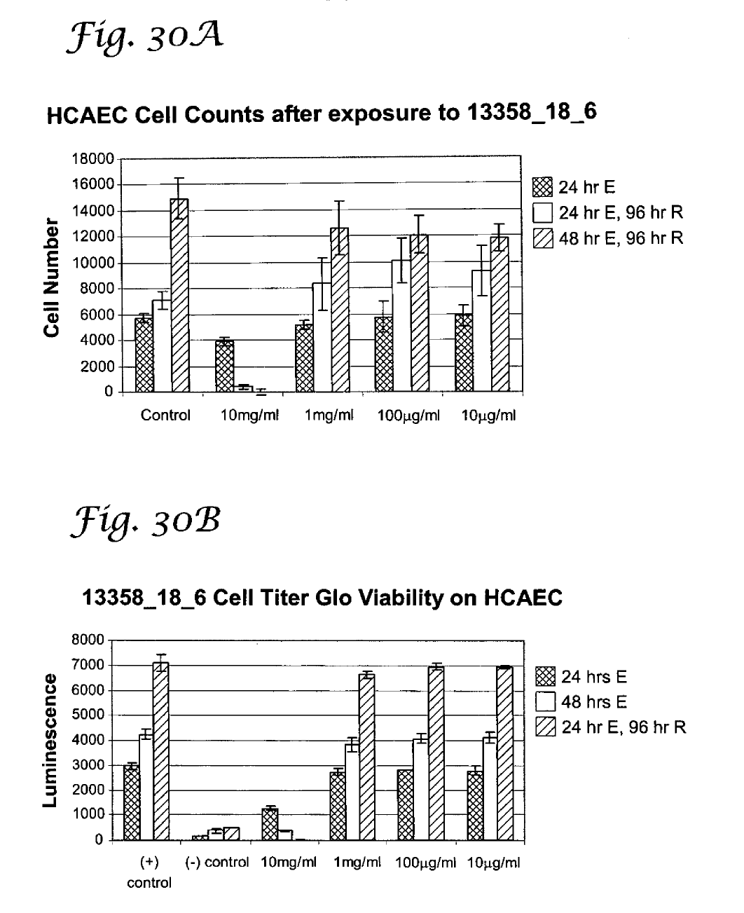

FIGS. 30(a) and (b) are graphical illustrations of cell counts and cell viability, respectively, for human coronary artery endothelial cells (HCAEC) exposed to 5,6-dihydroxy-hexan-2-one (13358-18-6) for 24 hours and 48 hours, then allowed to grow for 96 hours after removal of 5,6-dihydroxy-hexan-2-one.

Judged by the cell number following test compound exposure, the results indicated antiproliferative response to all agents at concentrations of 50 mg/ml and 10 mg/ml. The CelITiter-Glo assay suggested low cell viability at these conditions. These effects were present following 24 hours of agent exposure and persisted at 96 hours post treatment with the test compounds (96 hours without the test compounds). Compounds 13358-18-1, 13358-18-4, and 13358-18-5, when evaluated at 1 mg/ml showed some antiproliferative effect with viable cells after 96 hours post agent treatment but not following 24 hours exposure only.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of inhibiting or suppressing cellular proliferation, the method comprising delivering into or proximate a cell at least one antiproliferative agent selected from the group consisting of:
a compound of the formula (Formula I): 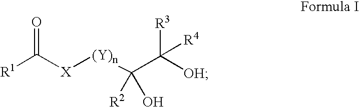
a compound of the formula (Formula II): 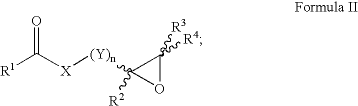
a compound of the formula (Formula III): 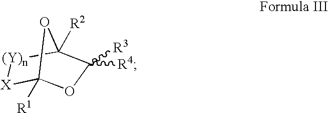
and combinations thereof;
wherein:
each X independently represents NR⁵, CR⁵R⁶, SiR⁵R⁶, S, a sulfur-bonded group, a phosphorus-bonded group, or 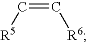
each Y independently represents O, NR⁵, CR⁵R⁶, SiR⁵R⁶, S, a sulfur-bonded group, a phosphorus-bonded group, or 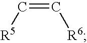
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.

2. The method of claim 1 wherein:
each X and Y independently represents CR⁵R⁶;
each n is 1;
each R¹ independently represents a C1-C10 organic group;
each R², R³, R⁴, R⁵, and R⁶ independently represents H or a C1-C10 organic group; and
R¹ and R⁵ can optionally be joined to each other to form a ring.

3. The method of claim 2 wherein:
each R¹ independently represents a C1-C4 aliphatic or alicyclic group;
each R², R³, R⁴, R⁵, and R⁶ independently represents H, a phenyl group, or a C1-C4 aliphatic or alicyclic group; and
R¹ and R⁵ can optionally be joined to each other to form a five- or six-membered ring.

4. The method of claim 2 wherein:
each R¹ independently represents a phenyl group;
each R², R³, R⁴, R⁵, and R⁶ independently represents H, a phenyl group, or a C1-C4 aliphatic or alicyclic group; and
R¹ and R⁵ can optionally be joined to each other to form a five- or six-membered ring.

5. The method of claim 1 wherein delivering into or proximate the cell further comprises:
providing a polymer having the at least one antiproliferative agent disposed therein;
locating the polymer proximate the cell; and
allowing the polymer to deliver the at least one antiproliferative agent into or proximate the cell.

6. The method of claim 5 wherein allowing the polymer to deliver the at least one antiproliferative agent comprises delivery of the at least one antiproliferative agent from pores in the polymer, diffusion of the at least one antiproliferative agent through the polymer, delivery of the at least one antiproliferative agent through degradation of the polymer, or combinations thereof.

7. The method of claim 1 wherein the cell is selected from the group consisting of fibroblast cells, smooth muscle cells, tumor cells, endothelial cells, and combinations thereof.

8. The method of claim 1 wherein the method is a method of treating or preventing a condition selected from the group consisting of treat or prevent conditions selected from the group consisting of hyperplasia of soft or hard tissues, tumor growth, skin keloids, fibrosis, surgical adhesions, in-stent restenosis, pannus overgrowth on prosthetic heart valves, urethral stenosis, stenosis associated with surgical anastomosis, occlusion of catheters, and combinations thereof.

9. A method of inhibiting or suppressing cellular proliferation, the method comprising:
locating at least one polymer proximate a tissue;
allowing the at least one polymer to hydrolyze to form at least one antiproliferative agent; and
delivering the at least one antiproliferative agent into or proximate a cell, wherein the at least one antiproliferative agent is selected from the group consisting of:
a compound of the formula (Formula I): 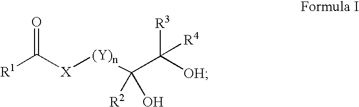
a compound of the formula (Formula II): 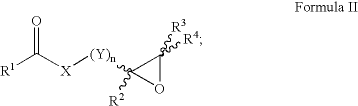
a compound of the formula (Formula III): 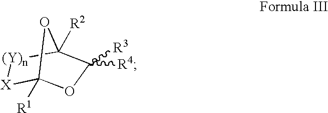
and combinations thereof;
wherein:
each X independently represents NR⁵, CR⁵R⁶, SiR⁵R⁶, S, a sulfur-bonded group, a phosphorus-bonded group, or 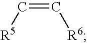
each Y independently represents O, NR⁵, CR⁵R⁶, SiR⁵R⁶, S, a sulfur-bonded group, a phosphorus-bonded group, or 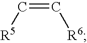
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.

10. The method of claim 9 wherein the at least one polymer is selected from the group consisting of polyesters, polyorthoesters, polyketals, and combinations thereof.

11. The method of claim 9 wherein the at least one polymer comprises two or more repeat units selected from the group consisting of:
a repeat unit of the formula (Formula IV): 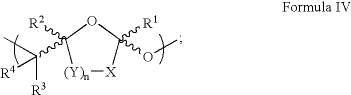
a repeat unit of the formula (Formula V): 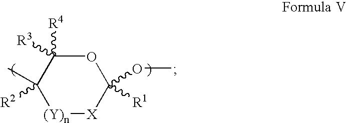
and
combinations thereof;
wherein:
each X independently represents NR⁵, CR⁵R⁶, SiR⁵R⁶, S, a sulfur-bonded group, a phosphorus-bonded group, or 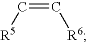
each Y independently represents O, NR⁵, CR⁵R⁶, SiR⁵R⁶, S, a sulfur-bonded group, a phosphorus-bonded group, or 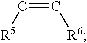
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.

12. The method of claim 11 wherein:
each X and Y independently represents CR⁵R⁶;
each n is 1;
each R¹ independently represents a C1-C10 organic group;
each R², R³, R⁴, R⁵, and R⁶ independently represents H or a C1-C10 organic group; and
R¹ and R⁵ can optionally be joined to each other to form a ring.

13. The method of claim 12 wherein:
each R¹ independently represents a C1-C4 aliphatic or alicyclic group;
each R², R³, R⁴, R⁵, and R⁶ independently represents H, a phenyl group, or a C1-C4 aliphatic or alicyclic group; and
R¹ and R⁵ can optionally be joined to each other to form a five- or six-membered ring.

14. The method of claim 12 wherein:
each R¹ independently represents a phenyl group;
each R², R³, R⁴, R⁵, and R⁶ independently represents H, a phenyl group, or a C1-C4 aliphatic or alicyclic group; and
R¹ and R⁵ can optionally be joined to each other to form a five- or six-membered ring.

15. The method of claim 11 wherein the at least one polymer further comprises repeat units selected from the group consisting of crosslinkable repeat units, crosslinked repeat units, repeat units having imagable groups, repeat units having latent reactive sites, and combinations thereof.

16. The method of claim 11 wherein the at least one polymer further comprises repeat units selected from the group consisting of alpha-hydroxy alkanoates, beta-hydroxy alkanoates, gamma-hydroxy alkanoates, delta-hydroxy alkanoates, epsilon-hydroxy alkanoates, glycols, carbonates, acetals, and combinations thereof.

17. The method of claim 11 wherein the polymer is a copolymer selected from the group consisting of random copolymers, alternating copolymers, block copolymers, graft copolymers, and combinations thereof.

18. The method of claim 17 wherein the copolymer is a block copolymer, and at least one block of the block copolymer is a polyketal block comprising the two or more repeat units selected from the group consisting of repeat units of Formula IV, repeat units of Formula V, and combinations thereof.

19. The method of claim 18 wherein at least one other block of the block copolymer includes repeat units selected from the group consisting of alpha-hydroxy alkanoates, beta-hydroxy alkanoates, gamma-hydroxy alkanoates, delta-hydroxy alkanoates, epsilon-hydroxy alkanoates, carbonates, acetals, and combinations thereof.

20. The method of claim 18 wherein at least one other block of the block copolymer is a polyorthoester block.

21. The method of claim 18 wherein at least one other block of the block copolymer is a poly(alkyleneglycol) block comprising alkylene glycol repeat units.

22. A method of inhibiting or suppressing cellular proliferation, the method comprising:
providing a medical device comprising at least one biodegradable polymer;
positioning the at least one biodegradable polymer proximate a tissue;
allowing the at least one biodegradable polymer to biodegrade to form at least one antiproliferative agent; and
delivering the at least one antiproliferative agent into or proximate a cell, wherein the at least one antiproliferative agent is selected from the group consisting of:
a compound of the formula (Formula I): 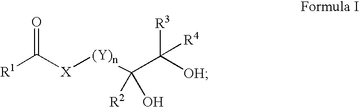
a compound of the formula (Formula II): 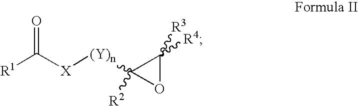
a compound of the formula (Formula III): 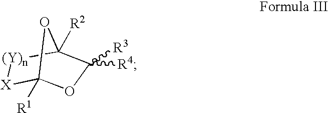
and combinations thereof;
wherein:
each X independently represents NR⁵, CR⁵R⁶, SiR⁵R⁶, S, a sulfur-bonded group, a phosphorus-bonded group, or 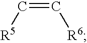
each Y independently represents O, NR⁵, CR⁵R⁶, SiR⁵R⁶, S, a sulfur-bonded group, a phosphorus-bonded group, or 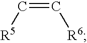
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.

23. The method of claim 22 wherein the medical device further comprises a biologically active agent different than the at least one antiproliferative agent disposed in the at least one biodegradable polymer.

24. A method of inhibiting or suppressing cellular proliferation, the method comprising delivering into or proximate a cell at least one antiproliferative agent selected from the group consisting of:
a ketal and/or a hemiketal of a compound of the formula (Formula I): 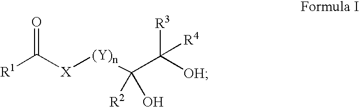
a ketal and/or a hemiketal of a compound of the formula (Formula II): 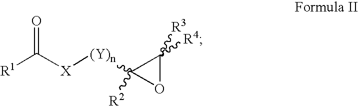
and combinations thereof;
wherein:
each X independently represents NR⁵, CR⁵R⁶, SiR⁵R⁶, S, a sulfur-bonded group, a phosphorus-bonded group, or 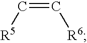
each Y independently represents O, NR⁵, CR⁵R⁶, SiR⁵R⁶, S, a sulfur-bonded group, a phosphorus-bonded group, or 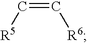
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.

25. A method of inhibiting or suppressing cellular proliferation, the method comprising delivering into or proximate a cell at least one prodrug that can release an antiproliferative agent selected from the group consisting of:
a compound of the formula (Formula I): 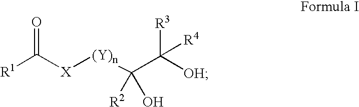
a compound of the formula (Formula II): 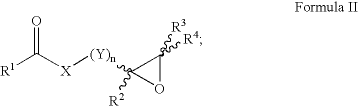
a compound of the formula (Formula III): 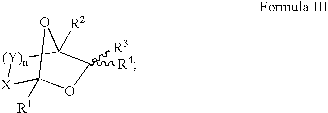
and combinations thereof;
wherein:
each X independently represents NR⁵, CR⁵R⁶, SiR⁵R⁶, S, a sulfur-bonded group, a phosphorus-bonded group, or 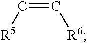
each Y independently represents O, NR⁵, CR⁵R⁶, SiR⁵R⁶, S, a sulfur-bonded group, a phosphorus-bonded group, or 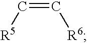
each n is independently from 0 to 5;
each R¹ independently represents an organic group;
each R², R³, R⁴, R⁵, and R⁶ independently represents H or an organic group; and
R¹, R², R³, R⁴, R⁵, and/or R⁶ can optionally be joined to each other to form one or more rings.
